(19) United States
(12) Patent Application Publication
(10) Pub. No.: US 2019/0017981 A1
(43) Pub. Date: Jan. 17, 2019

(54) SENSORS EMPLOYING A P-N SEMICONDUCTING OXIDE HETEROSTRUCTURE AND METHODS OF USING THEREOF

(71) Applicant: OHIO STATE INNOVATION FOUNDATION, Columbus, OH (US)

(72) Inventors: Prabir K. DUTTA, Worthington, OH (US); Chenhu SUN, Columbus, OH (US)

(21) Appl. No.: 15/781,460

(22) PCT Filed: May 19, 2016
(86) PCT No.: PCT/US2016/033326

§ 371 Date: Jun. 4, 2018

Related U.S. Application Data
(60) Provisional application No. 62/262,067, filed on Dec. 2, 2015.

(51) Int. Cl. G01N 33/00; G01N 27/12; G01N 27/30; G01N 27/403; G01N 33/497

(52) U.S. Cl. CPC G01N 33/0054; G01N 27/129; G01N 2033/4975; G01N 27/403; G01N 33/497; G01N 27/30

(57) ABSTRACT

Disclosed herein are p-n metal oxide semiconductor (MOS) heterostructure-based sensors and systems. The sensors and systems described herein can include sensing element that comprises a first region comprising a p-type MOS material (e.g., NiO) and a second region comprising an n-type MOS material (e.g., In₂O₃). These sensors and systems can exhibit sensitivity and selectivity to NH₃ at ppb levels, while discriminating against CO, NO, or a combination thereof at concentrations a thousand-fold higher (ppm) and spread over a considerable range (0-20 ppm). These sensors and systems can be used to detect and/or quantify NH3 in samples, including biological samples (e.g., breath samples) and combustion gases.

### CROSS REFERENCE TO RELATED APPLICATIONS

This application claims the benefit of U.S. Provisional Patent Application Ser. No. 62/262,067 filed Dec. 2, 2015, the disclosure of which is expressly incorporated herein by reference.

### BACKGROUND

Ammonia gas present in atmosphere at ppb-levels arises primarily from a variety of anthropogenic sources, such as combustion of fossil fuels, from use of fertilizes and metabolic activities. Since exposure to ammonia can cause health effects, there is a need for detection of ammonia in the environment. Ammonia is also produced in the human body and monitoring of ammonia in exhaled human breath can be correlated with several physiological conditions for disease diagnosis. The normal physiological range of breath ammonia is in the region of 50 to 2000 ppb. Each human breath contains over 1,000 trace volatile organic compounds, which makes breath a highly complex substance. Developing sensors for low level ammonia in the environment and human breath is a challenging problem because of the ppb sensitivity that is required and discrimination against other gases present at much higher concentrations.

### SUMMARY

Provided herein are p-n metal oxide semiconductor (MOS) heterostructure-based sensors and systems. The sensors and systems can be used for the detection and/or quantification of ammonia in a gas sample, such as a breath sample, an environmental sample, or a sample of combustion gas. In some cases, the sensors and systems described herein can be used for the detection and/or quantification of ammonia at concentrations of 5000 ppb or less (e.g., at concentrations of from 50 ppb to 2,000 ppb, at concentrations of from 50 ppb to 1,000 ppb, or at concentrations of from 50 ppb to 500 ppb). The sensors and systems can be used for the detection and/or quantification of ammonia in the presence of other gases, such as carbon monoxide and nitric oxide.

In some cases, the sensors and systems can be used to detect and/or quantify ammonia in the presence of one or more hydrocarbons, such as an aromatic hydrocarbon (e.g., toluene, o-xylene, or a combination thereof), an aliphatic hydrocarbon (e.g., hexane, pentane, isoprene, 3-methylpentane, or a combination thereof), a functional organic compound (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the sensors and systems can be used to detect and/or quantify ammonia at concentrations of 5000 ppb or less (e.g., at concentrations of from 50 ppb to 2,000 ppb, at concentrations of from 50 ppb to 1,000 ppb, or at concentrations of from 50 ppb to 500 ppb) in the presence of one or more hydrocarbons (e.g., one or more hydrocarbons at a concentration of from 50 ppb to 5 ppm), such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof).

Devices for sensing ammonia in a gas sample can comprise a sensing element that comprises a first region comprising a p-type metal oxide semiconductor (MOS) material and a second region comprising an n-type MOS material. The first region is adjacent to and contacts the second region (e.g., at a diffuse p-n heterojunction formed at an interface between the first and second regions). The p-type MOS material can comprise NiO. In certain embodiments, the p-type MOS material can consist of NiO. The n-type MOS material can comprise In₂O₃. In certain embodiments, the n-type MOS material can consist of In₂O₃.

In other embodiments, the p-type MOS material can be chosen from Co₃O₄, Cr₂O₃, Mn₃O₄, or a combination thereof; and the n-type MOS material chosen from ZnO, WO₃, SnO₂, TiO₂, Fe₂O₃, or a combination thereof. In other embodiments, the p-type MOS material does not include NiO and the n-type MOS material does not include In₂O₃.

The sensor device can further comprise one or more electrodes established and spaced apart within the first region and one or more electrodes established and spaced apart within the second region. In some embodiments, the sensor device can comprise a first electrode established within the first region, a second electrode established within the second region, and wiring interconnecting the first and second electrodes. A measured resistance along the wiring can be indicative of the presence of NH₃ in a gas interfacing with the sensing element.

In some embodiments, the location of the first electrode relative to the first region and the location of the second electrode relative to the second region are selected such that the measured resistance is unaffected by the presence of a gas other than ammonia (e.g., an interfering gas such as CO, NO, a hydrocarbon, or a combination thereof) that is also present the gas sample interfacing with the sensing element.

In some cases, the location of the first electrode relative to the first region and the location of the second electrode relative to the second region are selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the location of the first electrode relative to the first region and the location of the second electrode relative to the second region are selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensing element defines a length from a first side to an opposing second side, the first side being defined by an edge of the first region opposite the second region, the second side being defined by an edge of the second region opposite the first region, and the location of the first electrode relative to the first region and the location of the second electrode relative to the second region are selected such that the wiring encompasses a combined amount of the p-type MOS material and the n-type MOS material in the length direction that is pre-determined to generate a measured resistance indicative of the presence of NH₃ in a gas sample interfacing with the sensing element. The pre-determined combined amount can be selected such that the measured resistance is unaffected by the presence of a gas other than ammonia (e.g., an interfering gas such as CO, NO, a hydrocarbon, or a combination thereof) that is also present the gas sample interfacing with the sensing element. In some cases, the pre-determined combined amount can be selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the pre-determined combined amount can be selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensor device can further comprise a third electrode established within the first region, a fourth electrode established within the second region, and wiring interconnecting the third and fourth electrodes. A measured resistance along the wiring interconnecting the third and fourth electrodes in comparison with the measured resistance along the wiring interconnecting the first and second electrodes is indicative of a concentration of NH₃ in a gas interfacing with the sensing element.

In some embodiments, the device can further comprise a platform assembly maintaining the first and second electrodes as part of an electrode lead array selectively contacting the sensing element. The platform assembly can be configured to selectively alter a location of contact of the first electrode within the first region and selectively alter a location of contact of the second electrode within the second region. The platform assembly can be configured to selectively alter a distance between the first electrode and the second electrode.

Also provided are sensor systems for sensing ammonia in a gas sample. The sensor system can comprise a sensor device that comprises a sensing element, a first electrode established within the first region, a second electrode established within the second region, and a database. The sensing element can comprise a first region comprising a p-type MOS material and a second region comprising an n-type MOS material. The first region is adjacent to and contacts the second region (e.g., at a diffuse p-n heterojunction formed at an interface between the first and second regions). The p-type MOS material can comprise NiO. In certain embodiments, the p-type MOS material can consist of NiO. The n-type MOS material can comprise In₂O₃. In certain embodiments, the n-type MOS material can consist of In₂O₃. In other embodiments, the p-type MOS material can be chosen from Co₃O₄, Cr₂O₃, Mn₃O₄, or a combination thereof; and the n-type MOS material chosen from ZnO, WO₃, SnO₂, TiO₂, Fe₂O₃, or a combination thereof. In other embodiments, the p-type MOS material does not include NiO and the n-type MOS material does not include In₂O₃.

In certain embodiments, the system can be configured to estimate the concentration of NH₃ in a biological sample, such as human breath. For example, the system can be configured to detect and/or quantify ammonia at concentrations of 5000 ppb or less (e.g., at concentrations of from 50 ppb to 2,000 ppb, at concentrations of from 50 ppb to 1,000 ppb, or at concentrations of from 50 ppb to 500 ppb) in a sample of human breath. In other embodiments, the system can be configured to estimate the concentration of NH₃ a combustion gas. In other embodiments, the system can be configured to estimate the concentration of NH₃ an environmental sample.

The database can correlate measured resistance along wiring between the first electrode and the second electrode with presence of NH₃ in a gas sample interfacing with the sensing element. In some embodiments, the database can further correlate an estimate of a concentration of NH₃ in the gas sample based upon the measured resistance. In certain embodiments, the database can comprise a calibration curve for NH₃.

In some embodiments, the location of the first electrode relative to the first region and the location of the second electrode relative to the second region are selected such that the measured resistance is unaffected by the presence of a gas other than ammonia (e.g., an interfering gas such as CO, NO, a hydrocarbon, or a combination thereof) that is also present the gas sample interfacing with the sensing element. In some cases, the location of the first electrode relative to the first region and the location of the second electrode relative to the second region are selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the location of the first electrode relative to the first region and the location of the second electrode relative to the second region are selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensing element defines a length from a first side to an opposing second side, the first side being defined by an edge of the first region opposite the second region, the second side being defined by an edge of the second region opposite the first region, and the location of the first electrode relative to the first region and the location of the second electrode relative to the second region are selected such that the wiring encompasses a combined amount of the p-type MOS material and the n-type MOS material in the length direction that is pre-determined to generate a measured resistance indicative of the presence of NH₃ in a gas sample interfacing with the sensing element. The pre-determined combined amount can be selected such that the measured resistance is unaffected by the presence of a gas other than ammonia (e.g., an interfering gas such as CO, NO, a hydrocarbon, or a combination thereof) or a combination thereof, that is also present the gas sample interfacing with the sensing element. In some cases, the pre-determined combined amount can be selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the pre-determined combined amount can be selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensor device can further comprise a third electrode established within the first region, a fourth electrode established within the second region, and wiring interconnecting the third and fourth electrodes. A measured resistance along the wiring interconnecting the third and fourth electrodes in comparison with the measured resistance along the wiring interconnecting the first and second electrodes is indicative of a concentration of NH₃ in a gas interfacing with the sensing element.

In some embodiments, the sensor system can further comprise a controller maintaining the database and electronically associated with the wiring. The controller can comprise a memory on which is stored: the database; instructions for receiving a plurality of measured resistance values generated by the sensor device in the presence of the gas sample; and instructions for estimating a concentration of NH₃ in the gas sample based upon the plurality of measured resistances. In some embodiments, a first one of the plurality of measured resistances can correspond to a first distance between corresponding electrodes in the first and second regions, respectively, and a second one of the plurality of measured resistances can correspond to a second distance between corresponding electrodes in the first and second regions, respectively, the first distance being different from the second distance. The controller can further comprise a memory on which is stored instructions for performing appropriate resistance measurements to detect and/or quantify NH₃ in the gas sample. The controller can further comprise a memory on which is stored instructions for eliminating (e.g., subtracting or otherwise correcting for) the influence of a gas other than ammonia (e.g., an interfering gas such as CO, NO, a hydrocarbon, or a combination thereof) or a combination thereof, that is also present the gas sample interfacing with the sensing element. This can include, for example, calibration curve(s) for possible interferents (e.g., CO, NO, and/or one or more hydrocarbons) in the gas sample.

Optionally, in the case of systems configured to estimate the concentration of NH₃ in a biological sample such as human breath, the controller can comprise a memory on which is stored instructions for assigning a score for disease progression in a patient based on the estimated concentration of NH₃ in the gas sample associated with a biological sample from the patient (e.g., a breath sample from the patient). For example, the controller can comprise a memory on which is stored instructions for assigning a score for the progression of a liver disease in the patient, a kidney disease in the patient, an H. pylori infection in the patient, or halitosis in the patient. The score can be a numerical score assessing diseases progression or severity. Alternatively, the score can be a binary indicator of disease (e.g., a ‘positive’ or ‘negative’ indicator signifying the presence of an infection, such as an H. pylori infection). Optionally, in the case of systems configured to estimate the concentration of NH₃ in a biological sample such as human breath, the controller can comprise a memory on which is stored instructions for selecting one or more treatment instructions (e.g., one or more treatment options) based on the estimated concentration of NH₃ in the gas sample associated with a biological sample from the patient (e.g., a breath sample from the patient). The controller can comprise a memory on which is stored instructions for outputting these results to a person administering the test (e.g., the patient and/or a clinician). In this way, the sensors can be used as point-of-care diagnostic systems to assess the incidence and/or progression of a liver disease in a patient, a kidney disease in a patient, an H. pylori infection in a patient, and/or or halitosis in a patient.

Also provided are method of sensing ammonia using p-n MOS heterostructure-based sensors and systems. Methods can comprise providing a p-n MOS heterostructure-based sensor system; contacting the sensor element of the sensor system with the gas sample; measuring resistance along wiring between the first electrode and the second electrode, and detecting ammonia in the gas sample based upon the measured resistance. The sensor system can comprise a sensor device that comprises a sensing element, a first electrode established within the first region, a second electrode established within the second region, and a database. The sensing element can comprise a first region comprising a p-type MOS material and a second region comprising an n-type MOS material. The first region is adjacent to and contacts the second region (e.g., at a diffuse p-n heterojunction formed at an interface between the first and second regions). The p-type MOS material can comprise any suitable p-type MOS. In some cases, the p-type MOS material can comprise NiO, CuO, Co₃O₄, Cr₂O₃, Mn₃O₄, or a combination thereof. In some embodiments, the p-type MOS material can be chosen from NiO, Co₃O₄, Cr₂O₃, Mn₃O₄, or a combination thereof. In some embodiments, the p-type MOS material can be chosen from Co₃O₄, Cr₂O₃, Mn₃O₄, or a combination thereof. In some embodiments, the p-type MOS material can be chosen from NiO, CuO, or a combination thereof. In some embodiments, the p-type MOS material can comprise NiO. In certain embodiments, the p-type MOS material can consist of NiO. In other embodiments, the p-type material does not include NiO. The n-type MOS material can comprise any suitable n-type MOS. In some cases, the n-type MOS material can comprise In₂O₃, SnO₂, ZnO₂, TiO₂, WO₃, ZnO, Fe₂O₃, or a combination thereof. In some cases, the n-type MOS material can comprise In₂O₃, ZnO, WO₃, SnO₂, TiO₂, Fe₂O₃, or a combination thereof. In some cases, the n-type MOS material can comprise ZnO, WO₃, SnO₂, TiO₂, Fe₂O₃, or a combination thereof. In some cases, the n-type MOS material can comprise In₂O₃, SnO₂, ZnO₂, TiO₂, WO₃, or a combination thereof. In some embodiments, the n-type MOS material can comprise In₂O₃. In certain embodiments, the n-type MOS material can consist of In₂O₃. In other embodiments, the n-type material does not include In₂O₃. In one embodiment, the p-type MOS material does not include NiO and the n-type MOS material does not include In₂O₃.

The database can correlate measured resistance along wiring between the first electrode and the second electrode with presence of NH₃ in a gas sample interfacing with the sensing element. In some embodiments, the database can further correlate an estimate of a concentration of NH₃ in the gas sample based upon the measured resistance. In certain embodiments, the database can comprise a calibration curve.

In some embodiments, the location of the first electrode relative to the first region and the location of the second electrode relative to the second region are selected such that the measured resistance is unaffected by the presence of a gas other than ammonia (e.g., an interfering gas such as CO, NO, a hydrocarbon, or a combination thereof) that is also present the gas sample interfacing with the sensing element.

In some embodiments, the sensing element defines a length from a first side to an opposing second side, the first side being defined by an edge of the first region opposite the second region, the second side being defined by an edge of the second region opposite the first region, and the location of the first electrode relative to the first region and the location of the second electrode relative to the second region are selected such that the wiring encompasses a combined amount of the p-type MOS material and the n-type MOS material in the length direction that is pre-determined to generate a measured resistance indicative of the presence of NH₃ in a gas sample interfacing with the sensing element. The pre-determined combined amount can be selected such that the measured resistance is unaffected by the presence of a gas other than ammonia (e.g., an interfering gas such as CO, NO, a hydrocarbon, or a combination thereof) that is also present the gas sample interfacing with the sensing element.

In some embodiments, the sensor device can further comprise a third electrode established within the first region, a fourth electrode established within the second region, and wiring interconnecting the third and fourth electrodes. A measured resistance along the wiring interconnecting the third and fourth electrodes in comparison with the measured resistance along the wiring interconnecting the first and second electrodes is indicative of a concentration of NH₃ in a gas interfacing with the sensing element.

In some embodiments, the sensor system can further comprise a controller maintaining the database and electronically associated with the wiring. The controller can comprise a memory on which is stored: the database; instructions for receiving a plurality of measured resistance values generated by the sensor device in the presence of the gas sample; and instructions for estimating a concentration of NH₃ in the gas sample based upon the plurality of measured resistances. In some embodiments, a first one of the plurality of measured resistances can correspond to a first distance between corresponding electrodes in the first and second regions, respectively, and a second one of the plurality of measured resistances can correspond to a second distance between corresponding electrodes in the first and second regions, respectively, the first distance being different from the second distance.

Contacting the sensor element with the gas sample can comprise exposing the sensor element to the gas sample for a period of time effective to induce a change in the measured resistance along wiring between the first electrode and the second electrode. In some embodiments, contacting the sensor element with the gas sample comprises exposing the sensor element to the gas sample for a period of time effective to induce an change in resistance in the same direction in both the p-type MOS material and the n-type MOS material. In certain embodiments, contacting the sensor element with the gas sample comprises exposing the sensor element to the gas sample for a period of time effective to induce a decrease in the resistance of the p-type MOS material and a decrease in the resistance of the n-type MOS material. For example, contacting the sensor element with the gas sample can comprise exposing the sensor element to the gas sample for from 30 seconds to five minutes (e.g., for from 1 to 3 minutes).

In some embodiments, methods can further comprise heating the sensor element to a temperature of from 250° C. to 450° C. In some embodiments, detecting ammonia in the gas sample based upon the measured resistance comprises estimating a concentration of NH₃ in the gas sample based upon the measured resistance.

In some embodiments, the concentration of NH₃ in the gas sample can be 5,000 ppb or less (e.g., from 50 ppb to 2,000 ppb, from 50 ppb to 1,000 ppb, or from 50 ppb to 500 ppb). In some embodiments, the gas sample can comprise a biological sample, such as a human breath sample. In some embodiments, the gas sample can comprise a sample of a combustion gas, such as a sample of a combustion gas from a diesel engine. In some embodiments, the gas sample can comprise an environmental sample. In some embodiments, the gas sample can comprise a sample from an industrial process.

Also provided are sensor systems and methods for diagnosing an H. pylori infection in a patient. The sensor systems can comprise a sensor device that comprises a sensing element, a first electrode established within the first region, a second electrode established within the second region, and a database. The sensing element can comprise a first region comprising a p-type MOS material and a second region comprising an n-type MOS material. The first region is adjacent to and contacts the second region (e.g., at a diffuse p-n heterojunction formed at an interface between the first and second regions). The p-type MOS material can comprise NiO. In certain embodiments, the p-type MOS material can consist of NiO. The n-type MOS material can comprise In₂O₃. In certain embodiments, the n-type MOS material can consist of In₂O₃. In other embodiments, the p-type MOS material can be chosen from Co₃O₄, Cr₂O₃, Mn₃O₄, or a combination thereof; and the n-type MOS material chosen from ZnO, WO₃, SnO₂, TiO₂, Fe₂O₃, or a combination thereof. In other embodiments, the p-type MOS material does not include NiO and the n-type MOS material does not include In₂O₃.

In certain embodiments, the systems can be configured to estimate the concentration of NH₃ in a breath sample collected for a patient. For example, the system can be configured to detect and/or quantify ammonia at concentrations of 5000 ppb or less (e.g., at concentrations of from 50 ppb to 2,000 ppb, at concentrations of from 50 ppb to 1,000 ppb, or at concentrations of from 50 ppb to 500 ppb) in the breath sample. The system can further include a mouthpiece configured to receive a breath sample exhaled from a patient, and deliver the sample to the sensor device.

The database can correlate measured resistance along wiring between the first electrode and the second electrode with presence of NH₃ in a gas sample interfacing with the sensing element. In some embodiments, the database can further correlate an estimate of a concentration of NH₃ in the gas sample based upon the measured resistance. In certain embodiments, the database can comprise a calibration curve for NH₃.

In some embodiments, the location of the first electrode relative to the first region and the location of the second electrode relative to the second region are selected such that the measured resistance is unaffected by the presence of a gas other than ammonia (e.g., an interfering gas such as CO, NO, a hydrocarbon, or a combination thereof) that is also present the breath sample interfacing with the sensing element. In some cases, the location of the first electrode relative to the first region and the location of the second electrode relative to the second region are selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the location of the first electrode relative to the first region and the location of the second electrode relative to the second region are selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensing element defines a length from a first side to an opposing second side, the first side being defined by an edge of the first region opposite the second region, the second side being defined by an edge of the second region opposite the first region, and the location of the first electrode relative to the first region and the location of the second electrode relative to the second region are selected such that the wiring encompasses a combined amount of the p-type MOS material and the n-type MOS material in the length direction that is pre-determined to generate a measured resistance indicative of the presence of NH₃ in the breath sample interfacing with the sensing element. The pre-determined combined amount can be selected such that the measured resistance is unaffected by the presence of a gas other than ammonia (e.g., an interfering gas such as CO, NO, a hydrocarbon, or a combination thereof) or a combination thereof, that is also present the gas sample interfacing with the sensing element. In some cases, the pre-determined combined amount can be selected such that the measured resistance is unaffected by the presence of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the pre-determined combined amount can be selected such that the measured resistance is unaffected by the presence of from 50 ppb to 5 ppm of one or more hydrocarbons, such as one or more aromatic hydrocarbons (e.g., toluene, o-xylene, or a combination thereof), one or more aliphatic hydrocarbons (e.g., hexane, pentane, isoprene, 3-methylpentane, or a combination thereof), one or more functional organic compounds (e.g., acetone, acetonitrile, ethyl acetate, methyl vinyl ketone, ethanol, 2-methylfuran, hexanal, methacrolein, 1-propanol, 2-propanol, or a combination thereof), or a combination thereof.

In some embodiments, the sensor device can further comprise a third electrode established within the first region, a fourth electrode established within the second region, and wiring interconnecting the third and fourth electrodes. A measured resistance along the wiring interconnecting the third and fourth electrodes in comparison with the measured resistance along the wiring interconnecting the first and second electrodes is indicative of a concentration of NH₃ in the breath sample interfacing with the sensing element.

In some embodiments, the sensor systems can further comprise a controller maintaining the database and electronically associated with the wiring. The controller can comprise a memory on which is stored: the database; instructions for receiving a plurality of measured resistance values generated by the sensor device in the presence of the breath sample; and instructions for estimating a concentration of NH₃ in the breath sample based upon the plurality of measured resistances. In some embodiments, a first one of the plurality of measured resistances can correspond to a first distance between corresponding electrodes in the first and second regions, respectively, and a second one of the plurality of measured resistances can correspond to a second distance between corresponding electrodes in the first and second regions, respectively, the first distance being different from the second distance. The controller can further comprise a memory on which is stored instructions for performing appropriate resistance measurements to detect and/or quantify NH₃ in the breath sample.

The systems can further include a controller that comprises a memory on which is stored instructions for assigning a score for the progression of an H. pylori infection in the patient. The score can be a numerical score assessing the progression or severity of an H. pylori infection in the patient. Alternatively, the score can be a binary indicator of H. pylori infection (e.g., a ‘positive’ or ‘negative’ indicator signifying the presence of an H. pylori infection). In one embodiment, the instructions for assigning a score for the progression of an H. pylori infection can include instructions to provide a ‘positive’ indicator signifying the presence of an H. pylori infection in a patient when the estimated concentration of NH₃ in the breath sample is from 50 ppb to 400 ppb, and to provide a ‘negative’ indicator signifying the absence of an H. pylori infection in a patient when the estimated concentration of NH₃ in the breath sample is from 500 ppb to 600 ppb.

The systems can further include a controller that comprises a memory on which is stored instructions for performing appropriate resistance measurements to detect and/or quantify NH₃ in the control breath sample, instructions for receiving a plurality of measured resistance values generated by the sensor device in the presence of the control breath sample; and instructions for estimating a concentration of NH₃ in the control breath sample based upon the plurality of measured resistances. The systems can further include a controller that comprises a memory on which is stored instructions for subtracting the estimated concentration of NH₃ in the control breath sample from the estimated concentration of NH₃ in the breath sample. This can be used to determine the net change in the concentration of NH₃ in a patient's breath sample upon administration of urea.

In some cases systems can further include a controller that comprises a memory on which is stored instructions for assigning a score for the progression of an H. pylori infection in the patient based on the net change in the concentration of NH₃ in a patient's breath sample upon administration of urea. The score can be a numerical score assessing the progression or severity of an H. pylori infection in the patient. Alternatively, the score can be a binary indicator of H. pylori infection (e.g., a ‘positive’ or ‘negative’ indicator signifying the presence of an H. pylori infection). In one embodiment, the instructions for assigning a score for the progression of an H. pylori infection can include instructions to provide a ‘positive’ indicator signifying the presence of an H. pylori infection in a patient when the net change in the concentration of NH₃ in a patient's breath sample upon administration of urea is from 50 ppb to 400 ppb, and to provide a ‘negative’ indicator signifying the absence of an H. pylori infection in a patient when the net change in the concentration of NH₃ in a patient's breath sample upon administration of urea is from 500 ppb to 600 ppb.

Optionally, the controller can further comprise a memory on which is stored instructions for selecting one or more treatment instructions (e.g., one or more treatment options) based on the estimated concentration of NH₃ in the breath sample and/or the net change in the concentration of NH₃ in a patient's breath sample upon administration of urea. The controller can comprise a memory on which is stored instructions for outputting these results to a person utilizing the system to diagnose an H. pylori infection in a patient (e.g., the patient and/or a clinician). In this way, the systems can be used as point-of-care diagnostic systems to assess the incidence and/or progression of an H. pylori infection in a patient.

Methods for diagnosing an H. pylori infection in a patient can comprise administering urea (e.g., non-labeled urea) to a patient, collecting a breath sample from the patient, and measuring the concentration of NH₃ in the breath sample using the sensors and systems described herein. In one example, the concentration of NH₃ in the breath sample can be measured using a system described herein that is specifically configured to assess the incidence and/or progression of an H. pylori infection in a patient. Methods can further include collecting a control breath sample from the patient prior to administration of urea (e.g., non-labeled urea) to the patient, and measuring the concentration of NH₃ in the control breath sample using the sensors and systems described herein. In these cases, the methods can involve subtracting the estimated concentration of NH₃ in the control breath sample from the estimated concentration of NH₃ in the breath sample to determine the net change in the concentration of NH₃ in a patient's breath sample upon administration of urea. The net change in the concentration of NH₃ in a patient's breath sample upon administration of urea can be used to assess the incidence and/or progression of an H. pylori infection in a patient.

### DESCRIPTION OF DRAWINGS

FIG. 1A is plot of the x-ray diffraction pattern of NiO powder annealed at 320° C.

FIG. 1B is an SEM micrograph of NiO film on the sensor.

FIG. 1C is a plot of the XPS spectra of the Ni 2p (top) and O 1s (bottom) region for NiO powder annealed at 320° C.

FIG. 2A is plot of the x-ray diffraction pattern of In₂O₃ powder annealed at 320° C.

FIG. 2B is an SEM micrograph of In₂O₃ film on the sensor.

FIG. 2C is a plot of the XPS spectra of the Ni 2p (top) and O 1s (bottom) region for In₂O₃ powder annealed at 320° C.

FIG. 3 is a schematic representation of the multistep method used to fabricate sensors described herein.

FIG. 4A is a schematic diagram of sensors described herein.

FIG. 4B is a photograph of bare sensor substrate with four gold wires (left) and sensor with adjacent NiO and In₂O₃ (right).

FIG. 4C is a side view SEM image of the sensor.

FIG. 4D is a plot of the I-V characteristics across interface of NiO and In₂O₃ in 20% O₂/N₂ at 300° C., scan rate=0.1 V/s.

FIG. 5A is an SEM image of interface between NiO (top) and In₂O₃ (bottom).

FIG. 5B is a Raman spectra of NiO side.

FIG. 5C is a Raman spectra of In₂O₃ side.

FIG. 5D is a plot of the integrated Raman intensities by mapping from In₂O₃ side to NiO side (In₂O₃: straight line with square markers; NiO: dashed line with circle markers).

FIG. 6A is a plot of the gas sensing characteristics of NiO upon exposure to 1 ppm NH₃ at 300° C. with 10 min exposure time (20% O₂/N₂ as background).

FIG. 6B is a plot of the gas sensing characteristics of NiO upon exposure to 1 ppm NH₃ at 300° C. with 2 min exposure time (20% O₂/N₂ as background).

FIG. 6C is a plot of the gas sensing characteristics of NiO upon exposure to 1 ppm NH₃ at 500° C. with 10 min exposure time (20% O₂/N₂ as background).

FIG. 6D is a plot of the gas sensing characteristics of NiO upon exposure to 10 ppm NH₃ at 300° C. with 10 min exposure time (20% O₂/N₂ as background).

FIG. 7A is an in situ infrared spectra of NiO at 300° C. exposed to 1 ppm NH₃.

FIG. 7B is an in situ infrared spectra of NiO at 300° C. exposed to 10 ppm NH₃.

FIG. 7C is a plot of the relative peak height of the 1267 cm⁻¹ band for 1 ppm (straight line) and 10 ppm (dashed line) NH₃ as a function of time (in minutes).

FIG. 8A is a plot showing the gas sensing characteristics of sensing channel 1 (CH1, In₂O₃) for varying concentrations of CO at 300° C. (20% O₂/N₂ as background).

FIG. 8B is a plot showing the gas sensing characteristics of sensing channel 2 (CH2, NiO) for varying concentrations of CO at 300° C. (20% O₂/N₂ as background).

FIG. 8C is a plot showing the gas sensing characteristics of sensing channel 3 (CH3, In₂O₃—NiO) for varying concentrations of CO at 300° C. (20% O₂/N₂ as background).

FIG. 9A is a plot showing the gas sensing characteristics of sensing channel 1 (CH1, In₂O₃) for varying concentrations of NO at 300° C. (20% O₂/N₂ as background).

FIG. 9B is a plot showing the gas sensing characteristics of sensing channel 2 (CH2, NiO) for varying concentrations of NO at 300° C. (20% O₂/N₂ as background).

FIG. 9C is a plot showing the gas sensing characteristics of sensing channel 3 (CH3, In₂O₃—NiO) for varying concentrations of NO at 300° C. (20% O₂/N₂ as background).

FIG. 10A is a plot showing the gas sensing characteristics of sensing channel 1 (CH1, In₂O₃) for varying concentrations of NH₃ at 300° C. (20% O₂/N₂ as background).

FIG. 10B is a plot showing the gas sensing characteristics of sensing channel 2 (CH2, NiO) for varying concentrations of NH₃ at 300° C. (20% O₂/N₂ as background).

FIG. 10C is a plot showing the gas sensing characteristics of sensing channel 3 (CH3, In₂O₃—NiO) for varying concentrations of NH₃ at 300° C. (20% O₂/N₂ as background).

FIG. 11A is a plot showing the gas sensing characteristics of sensing channel 1 (CH1, In₂O₃) for varying concentrations of NH₃/CO mixture at 300° C. (20% O₂/N₂ as background).

FIG. 11B is a plot showing the gas sensing characteristics of sensing channel 2 (CH2, NiO) for varying concentrations of NH₃/CO mixture at 300° C. (20% O₂/N₂ as background).

FIG. 11C is a plot showing the gas sensing characteristics of sensing channel 3 (CH3, In₂O₃—NiO) for varying concentrations of NH₃/CO mixture at 300° C. (20% O₂/N₂ as background).

FIG. 12A is a schematic diagram illustrating a simulated breath system utilizing a 37° C. vapor bath.

FIG. 12B is a schematic diagram illustrating a simulated breath system utilizing a moisture trap with breath as the background.

FIG. 12C is a schematic diagram illustrating a simulated breath system utilizing a moisture trap with air as the background.

FIG. 13A is a plot showing the gas sensing characteristics of sensing channel 3 (CH3, In₂O₃—NiO) for a breath sample including varying concentrations of NH₃ at 300° C. obtained using a simulated breath system equipped with a 37° C. vapor bath.

FIG. 13B is a plot showing the gas sensing characteristics of sensing channel 3 (CH3, In₂O₃—NiO) for a breath sample including varying concentrations of NH₃ at 300° C. obtained using a simulated breath system equipped with an ice bath.

FIG. 13C is a plot showing the gas sensing characteristics of sensing channel 3 (CH3, In₂O₃—NiO) for a breath sample including varying concentrations of NH₃ at 300° C. obtained using a simulated breath system equipped with a dry ice/acetonitrile moisture trap.

FIG. 13D is a calibration curve for relative resistance changes (Ro/R) of sensing channel 3 (CH3, In₂O₃—NiO) for varying concentrations of NH₃ added to a breath sample (breath sample without spiked NH₃ used as background).

FIG. 14A is a plot showing the gas sensing characteristics of sensing channel 3 (CH3, In₂O₃—NiO) for breath sample B initially and then spiked with varying concentrations of NH₃ (10-1000 ppb) at 300° C. obtained using a simulated breath system equipped with a dry ice/acetonitrile moisture trap.

FIG. 14B is a calibration curve for relative resistance changes (Ro/R) of sensing channel 3 (CH3, In₂O₃—NiO) for varying concentrations of NH₃ in breath sample B (air as background).

FIG. 15 is a plot showing the gas sensing characteristics of all sensing channels (CH1 (In₂O₃), CH2 (NiO), CH3 (In₂O₃—NiO)) for a breath sample containing varying concentrations of NH₃ at 300° C. with 37° C. vapor bath.

FIG. 16 is a plot showing the gas sensing characteristics of all sensing channels (CH1 (In₂O₃), CH2 (NiO), CH3 (In₂O₃—NiO)) for breath sample containing varying concentrations of NH₃ at 300° C. with ice bath moisture trap.

FIG. 17 is a plot showing the gas sensing characteristics of all sensing channels (CH1 (In₂O₃), CH2 (NiO), CH3 (In₂O₃—NiO)) for breath sample containing varying concentrations of NH₃ at 300° C. with dry ice/acetonitrile moisture trap.

FIG. 18A is a plot of the infrared spectra of NiO exposed to NH₃ at 300° C. in an oxygen background and then cooled to room temperature.

FIG. 18B is a plot of the infrared spectra of NiO exposed to NH₃ at 300° C. in a N₂ background and then cooled to room temperature.

FIG. 19 is a schematic illustration of a sensor device and sensor system.

FIG. 20 is a schematic illustration of a sensor device and sensor system including electrodes.

FIG. 21 is a schematic illustration of a sensor device and sensor system including NiO and In₂O₃.

### DETAILED DESCRIPTION

Provided herein are sensor devices and corresponding sensor systems that employ a p-n semiconducting oxide heterojunction. The devices and systems described herein can be used to detect and/or quantify the amount of NH₃ in a gas sample. In some cases, the devices and systems described herein can be used to detect and/or quantify the amount of NH₃ in a gas sample in the presence of other gases such as CO, NO, or a combination thereof. The sensors described herein comprise p-type and n-type materials arranged adjacent one another, forming the sensing element of the sensor device. In this regard, techniques for obtaining data from the so-constructed sensor device can assist in distinguishing NH₃ from a mixture of gases, and allow for the detection and/or quantification of NH₃ in the presence of one or more interfering gases, such as CO, NO, or a combination thereof.

In some cases, the sensors and systems can be used to detect and/or quantify ammonia in the presence of one or more hydrocarbons, such as an aromatic hydrocarbon (e.g., toluene, o-xylene, or a combination thereof), an aliphatic hydrocarbon (e.g., hexane, pentane, isoprene, 3-methylpentane, or a combination thereof), a functional organic compound (e.g., acetone, acetonitrile, ethyl acetate, methyl vinyl ketone, ethanol, 2-methylfuran, hexanal, methacrolein, 1-propanol, 2-propanol, or a combination thereof), or a combination thereof. In certain embodiments, the sensors and systems can be used to detect and/or quantify ammonia at concentrations of 5000 ppb or less (e.g., at concentrations of from 50 ppb to 2,000 ppb, at concentrations of from 50 ppb to 1,000 ppb, or at concentrations of from 50 ppb to 500 ppb) in the presence of one or more hydrocarbons, such as an aromatic hydrocarbon (e.g., toluene, o-xylene, or a combination thereof), an aliphatic hydrocarbon (e.g., hexane, pentane, isoprene, 3-methylpentane, or a combination thereof), a functional organic compound (e.g., acetone, acetonitrile, ethyl acetate, methyl vinyl ketone, ethanol, 2-methylfuran, hexanal, methacrolein, 1-propanol, 2-propanol, or a combination thereof), or a combination thereof).

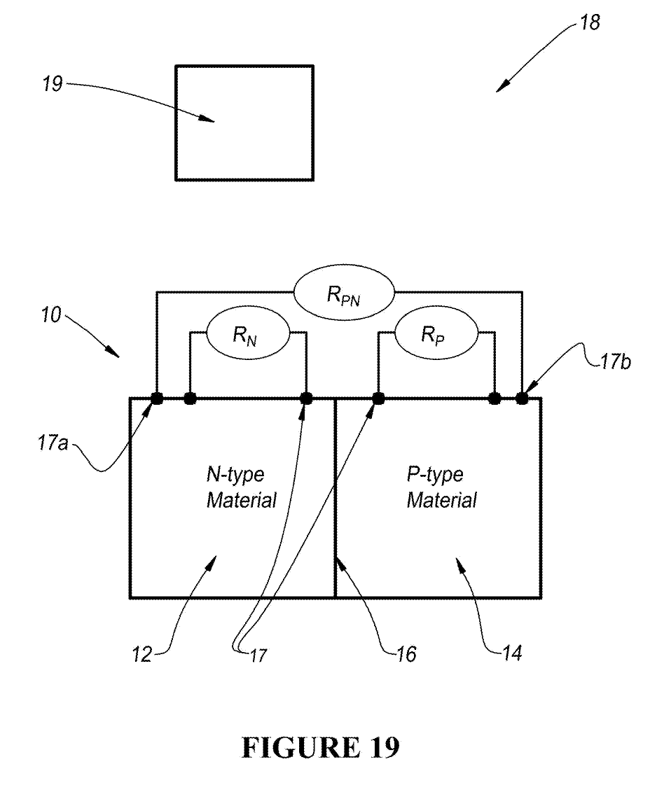

An example sensor device (10) is schematically illustrated in FIG. 19. The sensor device (10) can include a sensing element (11) akin to a MOS sensing element, but formed by at least two discrete MOS materials. Namely, the sensing element (11) includes a first, n-type MOS material region (12) and a second, p-type MOS material region (14). A diffuse p-n junction (16) can be established between the n-type region (12) and the p-type region (14). The n-type and p-type regions (12, 14) are formed immediately adjacent one another and can contact one another at the p-n junction (16). Electrodes or other electrical lead-type bodies (identified generally at 17) are, or can be, selectively or permanently established at nodes within each of the regions 12, 14 (e.g., gold electrodes provided with a gold microspring array (now shown)). Electrical connections (e.g., wires) can be established between selected pairs of the so-established electrodes or nodes (17), with FIG. 19 illustrating three possible connections as measured resistances R_{P}, R_{N}, and R_{PN}. R_{P} represents a measured resistance between two nodes (17) only in the p-type region (12). R_{N} represents a measured resistance between two nodes (17) only within the n-type region (14). R_{PN} represents a measured resistance spanning both the p- and n-type regions 12, 14 (e.g., the electrodes 17a and 17b of FIG. 19). In one embodiment, a platform (not shown) supports the sensor element (11) and can be maintained at a temperature optimized for the analyte.

The sensor device (10) can be provided as part of a sensor system (18) as described herein. The sensor system (18) can include components conventionally employed with MOS-type gas sensor systems, such as a housing (not shown) for directing a gas or other substance of interest across the sensing element 11, electronics for establishing and measuring conductivity at the desired connections (e.g., R_{P}, R_{N}, R_{PN}), and a controller 19 (e.g., a computer or other logic device) for receiving and/or interpreting the measured conductivity signals. In some embodiments, a measurement device (e.g., a multimeter) can be provided apart from the controller 19 that measures resistance at the selected connection(s), and signals the measured resistance value(s) to the controller 19 for interpretation as described below. The sensor system 18 can, in some embodiments, be provided as a single unit, such as a hand-held device providing an inlet port through which a gas sample is introduced. Regardless, the controller 19 can further programmed to determine the presence and amount (e.g., in ppm or ppb) of one or more analytes (e.g., ammonia) of interest based upon the measured conductivity signals. In certain embodiments, the controller 19 can be programmed to operate the sensor device 10 and analyze data generated thereby to detect the presence of, and estimate the concentration of, ammonia in various sample types, including human breath samples and combustion gas samples. In other embodiments, some or all of the measured resistance interpretation can be performed manually, such that the controller 19 can be optional.

The p-type material region 12 includes a p-type MOS material that conducts with positive holes being the majority charge carrier. Generally, in the presence of an oxidizing gas, the p-type MOS materials exhibit an increase in conductivity (or decrease in resistivity). An opposite effect is generally exhibited by the p-type MOS material in the presence of a reducing gas. However, in the case of NiO, a transient decrease in resistance upon exposure of low levels of NH₃ can be observed. This effect can be exploited to amplify the response of the sensors described herein towards ammonia. The p-type MOS material can comprise NiO. In certain embodiments, the p-type MOS material can comprise at least 75% wt NiO (e.g., at least 80% wt NiO, at least 85% wt NiO, at least 90% wt NiO, at least 95% wt NiO, at least 96% wt NiO, at least 97% wt NiO, at least 98% wt NiO, or at least 99% wt NiO), based on the total weight of the p-type MOS material. In certain embodiments, the p-type MOS material can consist of NiO.

The n-type material region (14) includes an n-type MOS material in which the majority charge carriers are electrons. Generally, upon interaction with an oxidizing gas, the n-type MOS material exhibits a decrease in conductivity (or increase in resistivity). An opposite effect is exhibited by the n-type MOS material in the presence of a reducing gas. The n-type MOS material can comprise In₂O₃. In certain embodiments, the n-type MOS material can comprise at least 75% wt In₂O₃ (e.g., at least 80% wt In₂O₃, at least 85% wt In₂O₃, at least 90% wt In₂O₃, at least 95% wt In₂O₃, at least 96% wt In₂O₃, at least 97% wt In₂O₃, at least 98% wt In₂O₃, or at least 99% wt In₂O₃), based on the total weight of the n-type MOS material. In certain embodiments, the n-type MOS material can consist of In₂O₃.

In other embodiments, the p-type MOS material can be chosen from NiO, Co₃O₄, Cr₂O₃, Mn₃O₄, or a combination thereof; and the n-type MOS material chosen from In₂O₃, ZnO, WO₃, SnO₂, TiO₂, Fe₂O₃, or a combination thereof. In certain embodiments, the p-type MOS material can be chosen from Co₃O₄, Cr₂O₃, Mn₃O₄, or a combination thereof; and the n-type MOS material chosen from ZnO, WO₃, SnO₂, TiO₂, Fe₂O₃, or a combination thereof. In certain embodiments, the p-type MOS material does not include NiO. In certain embodiments, the n-type MOS material does not include In₂O₃. In one embodiment, the p-type MOS material does not include NiO and the n-type MOS material does not include In₂O₃.

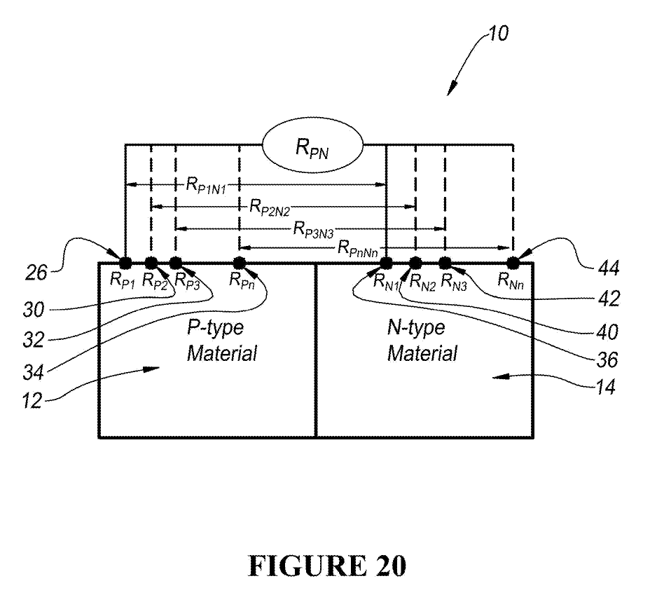

The measured conductivities at the p-type region R_{P}, at the n-type region R_{N}, and across the p-n junction R_{PN} can be evaluated to determine the presence and amount of a particular gas, such as ammonia, as different changes in conductivity are expected in each of these regions upon exposure to a gas such as NH₃. The signal analysis can assume various forms, and can include obtaining a multiplicity of p-n junction measurements at differing nodes within the p-type region and the n-type region. For example, FIG. 20 illustrates an alternative layout of leads or nodes (and corresponding electrical connections or wires) along the sensor device 10 and will help explain further the basis of the analyte identification based on a concept of cancellation. With a proper combination of the p-type material in the p-type region 12 and n-type material in the n-type region 14, using one of the lead wires from the electrodes or nodes at R_{P1} (26), R_{P2} (30), R_{P3} (32), R_{Pn} (34) in the p-type region 12 and other lead wires from the electrodes or nodes at R_{N1} (36), R_{N2} (40), R_{N3} (42), R_{Nn} (44) in the n-type region 14, the analyte signal may diminish completely and may be treated as null response for the particular analyte. Thus, different types of analyte molecules will have unique null response spacings. For example, a first analyte may have a null response spacing between R_{P1} (26) and R_{N1} (36), a second (different) analyte may have a null response spacing between R_{P2} (30) and R_{N2} (40), etc.

With the above in mind, it should be noted that the null response data can be used as a “fingerprint” signature that is unique to a specific analyte. Thus, in a blind study, sensors and systems can elucidate the identity of analytes using this “fingerprint” signature technique. For example, the controller 19 (FIG. 19) can be programmed to include a database of various analytes and their corresponding, previously-determined null response data; the controller 19 can compare the conductivity information (e.g., null spacing data) for an unknown analyte being tested with the database to identify the unknown analyte.

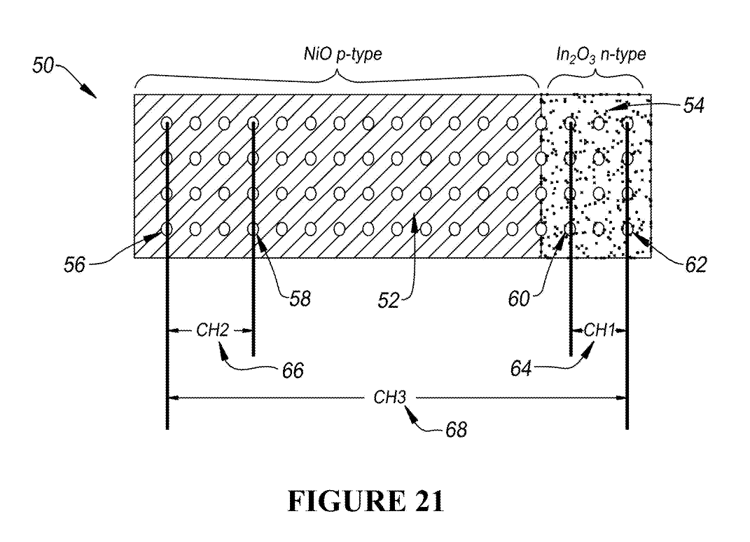

With these principles in mind, an example ammonia sensor incorporating NiO as the p-type material and In₂O₃ as the n-type material is schematically illustrated in FIG. 21. The sensor device 50 is schematically illustrated as including a p-type material 52 of NiO and n-type material 54 of In₂O₃, and is surprisingly found to have very high sensitivity to NH₃ and discrimination against CO and NO. Electrode wires are illustrated as extending from electrodes or nodes 56 and 58 in the p-type material 52, and extending from electrodes or nodes 60 and 62 in the n-type material 54. Channels 1, 2, and 3 (“CH 1”-“CH 3”) 64, 66, 68 are illustrated between the wire of the electrode 60 and the wire of the electrode 62, between the wire of the electrode 56 and the wire of the electrode 58, and between the wire of the electrode 56 and the wire of the electrode 62, respectively.

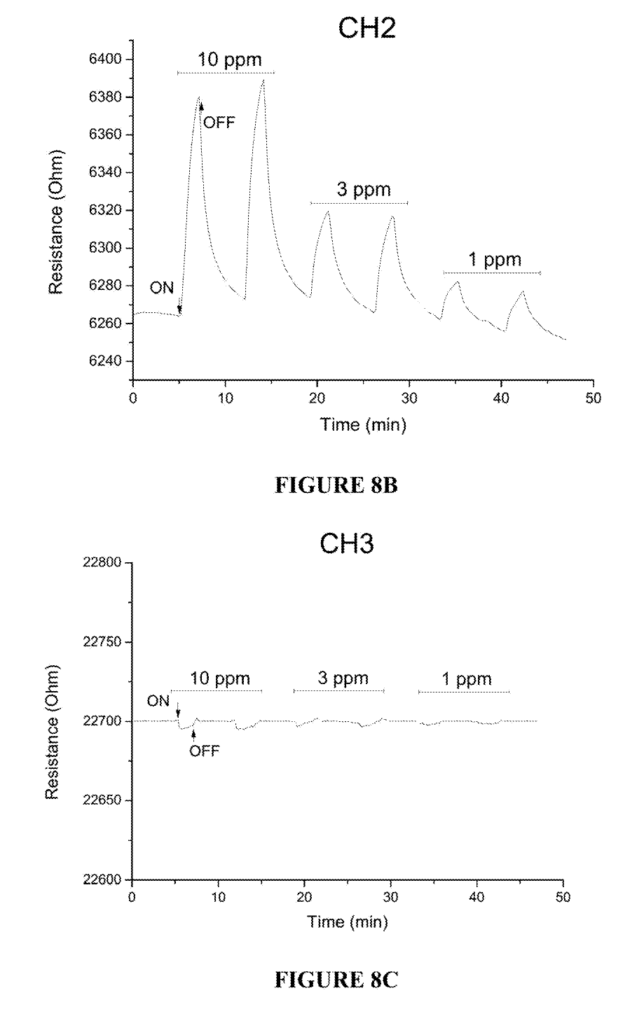

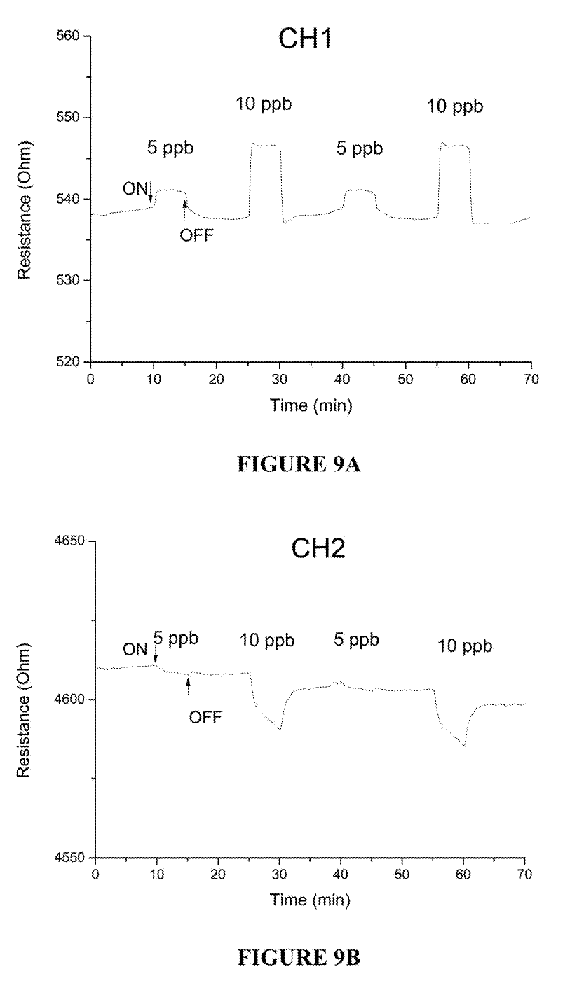

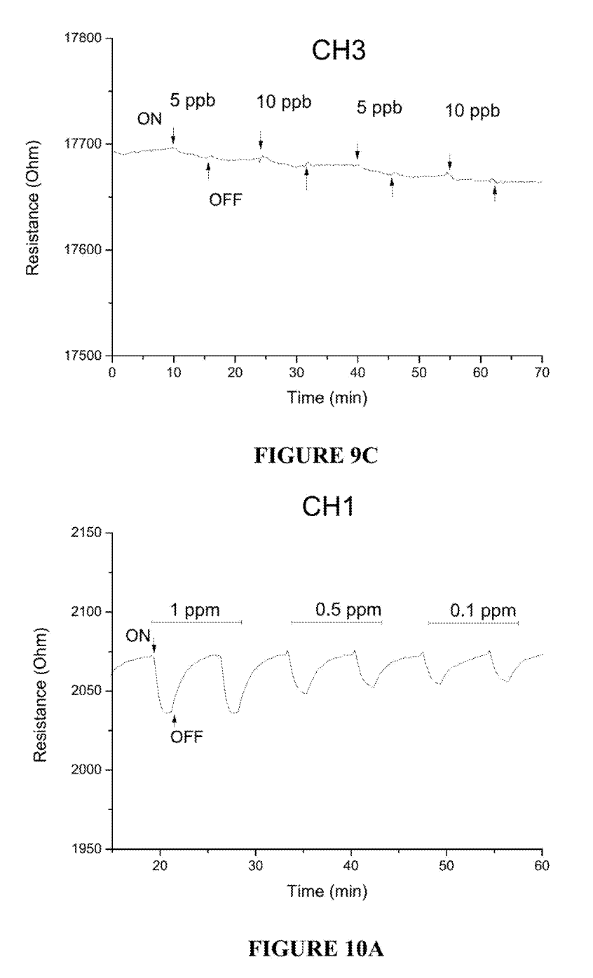

The measured resistance at each of the channels 64-68 differs in the presence of NH₃, NO, or CO, and varies as a function of the NH₃, NO, or CO concentrations. By way of example, FIG. 8A is a plot of the measured resistance obtained from Channel 1 64 in response to combinations of 20% O₂/N₂ with various concentrations of CO (1, 3 and 10 ppm CO). A decrease in resistance was observed at CO concentrations of 1, 3 and 10 ppm, with higher concentrations exhibiting a progressively decreasing signal. FIG. 8B is a plot of the measured resistance obtained from Channel 2 66 in response to combinations of 20% O₂/N₂ with various concentrations of CO (1, 3 and 10 ppm CO). An increase in resistance was observed at NO concentrations of 1, 3 and 10 ppm, with higher concentrations exhibiting a progressively increasing signal. FIGS. 8A and 8B can be contrasted with FIG. 8C, which shows a plot of the measured resistance obtained from Channel 3 68 in response to combinations of 20% O₂/N₂ with various concentrations of CO (1, 3 and 10 ppm CO). The CO signal at 1 and 3 ppm is completely nulled and a very small signal is observed for 10 ppm CO. Similar results were obtained for NO (see FIGS. 9A-9C).

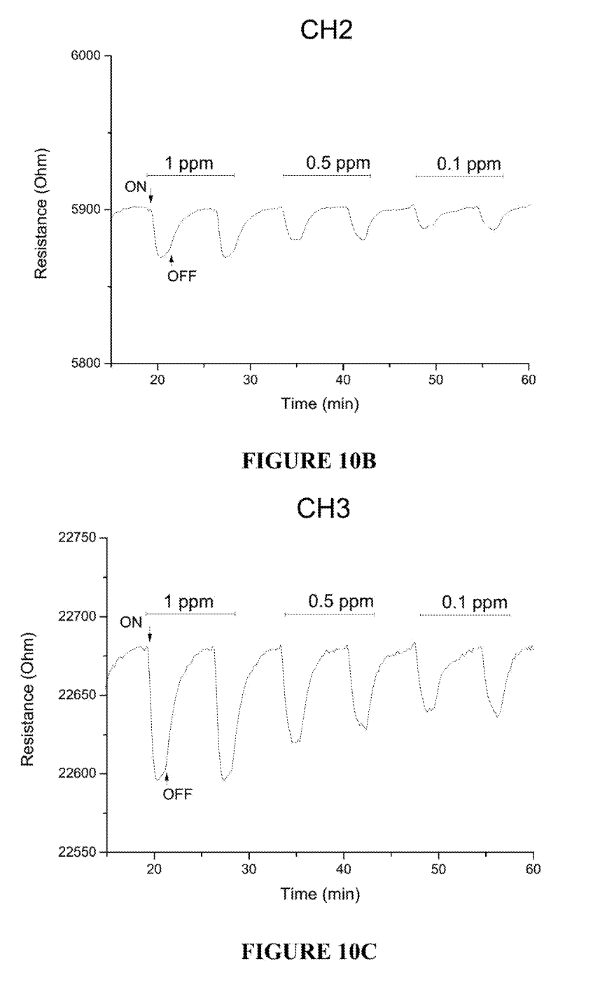

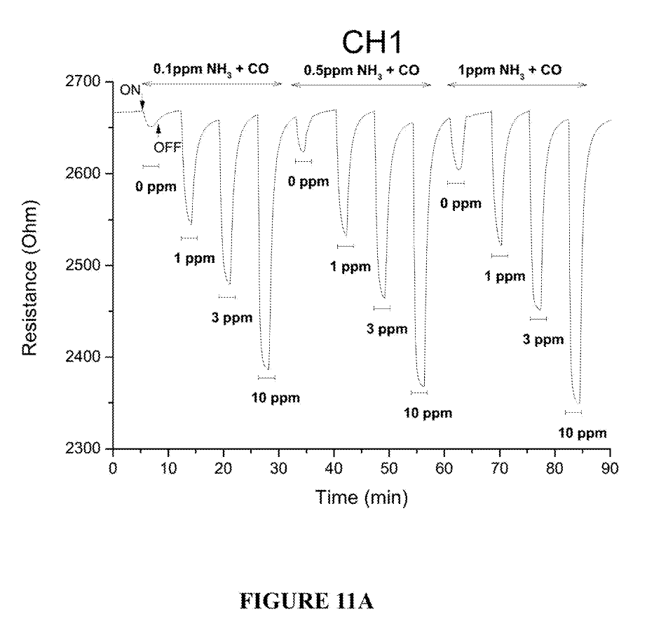

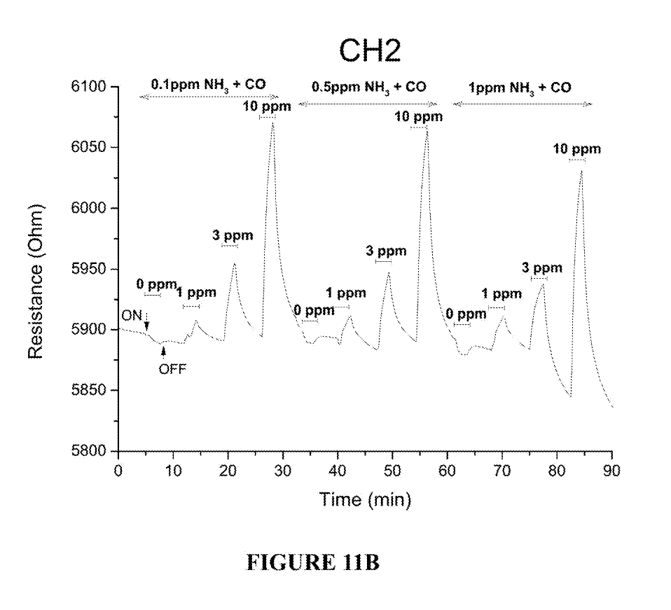

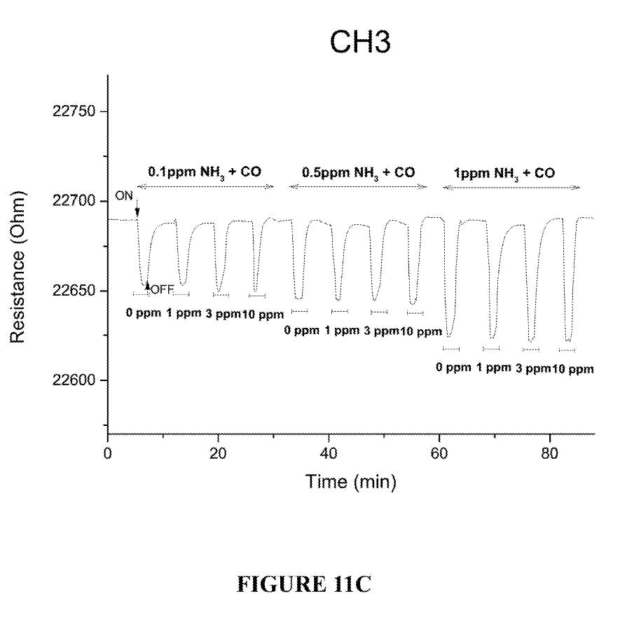

In the case of NH₃ (see FIGS. 10A-10C), a decrease in resistance was observed at NH₃ concentrations of 1 ppm, 0.5 ppm, and 0.1 ppm from all three channels when short pulses of sample gas (e.g., 2 minutes in length) were used, with higher concentrations exhibiting a progressively decreasing signal. Significantly, even at very low concentrations of NH₃ (e.g., 100 ppb), the response remains significant for Channel 3 68. The response upon exposure to NH₃ was heightened by exposing the sensor to NH₃ for relatively short periods of time as described in more detail in Example 1. For example, the sensor can be exposed to NH₃ for intervals of from 30 seconds to five minutes (e.g., for from 1 to 3 minutes, or for about 2 minutes). By exposing the sensor to NH₃ for brief intervals of time, both NiO and In₂O₃ (Channels 1 and 2) show a decrease in resistance, so sensing data that combines both oxides (Channel 3) exhibits an additive effect, amplifying the response from NH₃ (FIGS. 10A-10C) while with CO and NO, the opposite response lead to a cancellation of signal (FIGS. 8A-8C, 9A-9C). This strategy allows for the detection and/or quantification of NH₃ at concentrations <1000 ppb (e.g., from 50 ppb to 1000 ppb), in the presence of CO (and/or NO and/or hydrocarbons), as shown in FIGS. 11A-11C.

With the above explanations in mind, sensor devices (and corresponding sensor systems) can be used to effectively sense the presence and concentration of NH₃, including discriminating against the presence of CO and/or NO and/or hydrocarbons, as described above.

As described below, non-limiting examples of NH₃ sensor devices in accordance with certain embodiments of the present disclosure were constructed and subjected to testing to confirm viability in sensing NH₃, including sensing NH₃ in human breath.

### EXAMPLES

### Example 1: Selective Detection of Part Per Billion Concentrations of Ammonia Using a p-n Semiconducting Oxide Heterostructure

The detection of low levels of ammonia is relevant for environmental, combustion and health-related applications. Resistive semiconducting metal oxide sensing platforms can be used for ammonia and other gas detection. Two important aspects of gas sensing are enhancing sensitivity and selectivity. A sensor platform with n-type In₂O₃ and p-type NiO placed side-by-side with a shared 30 μm interface was investigated. The substrate on which these metal oxides are placed allows for measuring the resistance change across In₂O₃, NiO or any combination of both oxides. With low concentrations of NH₃ (<100 ppb), the change in resistance with NiO was anomalous at 300° C., the resistance decreased and then gradually increased over tens of minutes before decreasing again to reach the baseline. In situ diffuse reflectance infrared spectroscopy exhibited a band at 1267 cm⁻¹, which was assigned to O₂⁻ and the change in intensity of this band with time mirrored the transient change in resistance with 1 ppm NH₃ at 300° C., indicating that NH₃ chemisorption was correlated with the O₂⁻ species. Taking advantage of the transient resistance decrease of NiO with NH₃, and combining the In₂O₃ and NiO allowed selectivity enhancement towards NH₃ at concentrations as low as 100 ppb. Interference to CO, NOₓ and humidity were studied. By selecting a suitable combination of both oxides, the response to CO at <10 ppm could be negated. Similarly, with NO at <10 ppb, there was minimal sensor response. The sensor was used to analyze NH₃ mixed into human breath at 10-1000 ppb concentrations. Water had to be completely removed from the breath via a moisture trap, since water interfered with the NH₃ chemisorption chemistry. Potential applications of this sensor platform in breath analysis are discussed

Herein, ammonia sensors with ppb sensitivity, with possible application in breath analysis, were investigated.

### INTRODUCTION

Methods for measurement of ammonia (NH₃) are relevant to environmental, combustion and health related industries. Ammonia in the atmosphere arises primarily from anthropogenic sources, including agriculture (nitrogen fixation, ammonification) and emissions from chemical industry involved in development of refrigeration and fertilizers. Ammonia is a lachrymatory gas, and breathing ammonia at high concentrations (˜1000 ppm) can induce laryngospasms, and cause bronchiectasis. Thus, there is a need for environmental ammonia monitors. The transportation industry is also interested in measuring ammonia from exhaust emissions, air quality control in passenger compartment and in a new generation of lean-burn combustion engines, where the exhaust gas after-treatment includes reaction of nitrogen oxides with ammonia. Ammonia is also produced in the human body, and monitoring ammonia in exhaled human breath has potential applications in healthcare settings (e.g., for disease diagnosis). As an example, breath ammonia measurement can be used to probe several diseases including malfunctioning of liver and kidney, H. pylori infection, and halitosis. The concentration ranges over which ammonia detection is relevant for these applications range from 0.1 ppm (health) to hundreds of ppm (environmental).

Different measurement principles have been applied for the detection of ammonia, including optical spectroscopy, electrochemistry and wet-chemistry methods. A particularly challenging application is the detection of ammonia in human breath. Tunable diode laser absorption spectroscopy has been used to detect ammonia in breath, with detection limit of 1 ppm. A quantum cascade laser diode was able to measure ammonia as low as 4 ppb. Other strategies include use of quartz crystal microbalance and liquid-film conductive sensor. Sensors based on conducting polymer junctions can detect ppb ammonia in human breath, and a p-n heterojunction polyaniline-TiO₂ sensor is reported to have ppt sensitivity. Mass spectrometry also can measure ammonia down to ppb levels. Instruments for measuring ammonia are often bulky, and there is a drive for obtaining miniaturized sensors.

Solid state electrochemical sensors have been developed for monitoring ammonia. This technology is attractive since high sensitivity, selectivity, and fast response time are possible. In addition, these devices have advantages of low power consumption, light-weight, low maintenance cost, harsh environment tolerance and portability. There are numerous papers on resistive semiconducting metal oxide sensors for ammonia. The working principle of these devices is associated with the adsorption of gas molecules on the oxide's surface inducing charge transfer, which result in changes in resistance of the oxide. Semiconducting metal oxides such as n-type WO₃, SnO₂, In₂O₃, ZnO, TiO₂, MoO₃ as well as p-type Cr₂O₃, NiO, CuO, have been studied as sensing materials to detect NH₃. To promote sensitivity and selectivity, noble metals like Pt, Pd, Au, and Ag have been introduced to metal oxides. Of these, MoO₃-based sensors have been developed for measuring ammonia in human breath.

However, developing an electrochemical sensor platform that can measure low concentrations of ammonia in the environment, in optimizing combustion processes and in human breath is still a challenge. There is the need for ppb sensitivity, discrimination against other gases present at much higher concentrations, and in the case of combustion, the ability to tolerate harsh environments, and be insensitive to other exhaust gases.

Mixtures of p- and n-semiconducting oxides can improve sensor performance. Examples include anatase/rutile for CO detection, ZnO/NiO for NH₃ detection, In₂O₃/NiO for ethanol detection, and CuO/SnO₂ for H₂S detection. These designs are mixtures of p- and n-powders, or the p-type material grown on n-type powders and vice versa. In addition, isotype heterojunctions, prepared by mixing powders, such as WO₃ and ZnO have also shown selective gas sensing.

Provided herein is a sensor device that includes an adjacent alignment of p-type NiO and n-type In₂O₃ deposited on a gold microspring array. This semiconductor hetero-junction structure can be used for the detection of ammonia at ppb levels, while discriminating against nitric oxide at ppb levels, and carbon monoxide at significantly higher ppm concentrations. The potential application of detection of ammonia in human breath samples is also demonstrated, suggesting the application of this sensor platform in a future breath monitoring device.

### Experimental

Chemicals and Materials

Indium (II) oxide (99.99%, metals basis, ˜325 mesh powder), nickel (II) oxide (99.998%, metals basis), alpha-terpineol (96%), gold wires (0.127 mm dia, 99.99%) were purchase from Alfa Aesar (Ward Hill, USA). The plastic substrates with gold microspring arrays were obtained from FormFactor, Inc. (USA). The interdigitated electrodes were obtained from Case Western Reserve University. All test gases including nitrogen, oxygen, ammonia and carbon monoxide were supplied by Praxair (Danbury, USA).

Sensor Fabrication

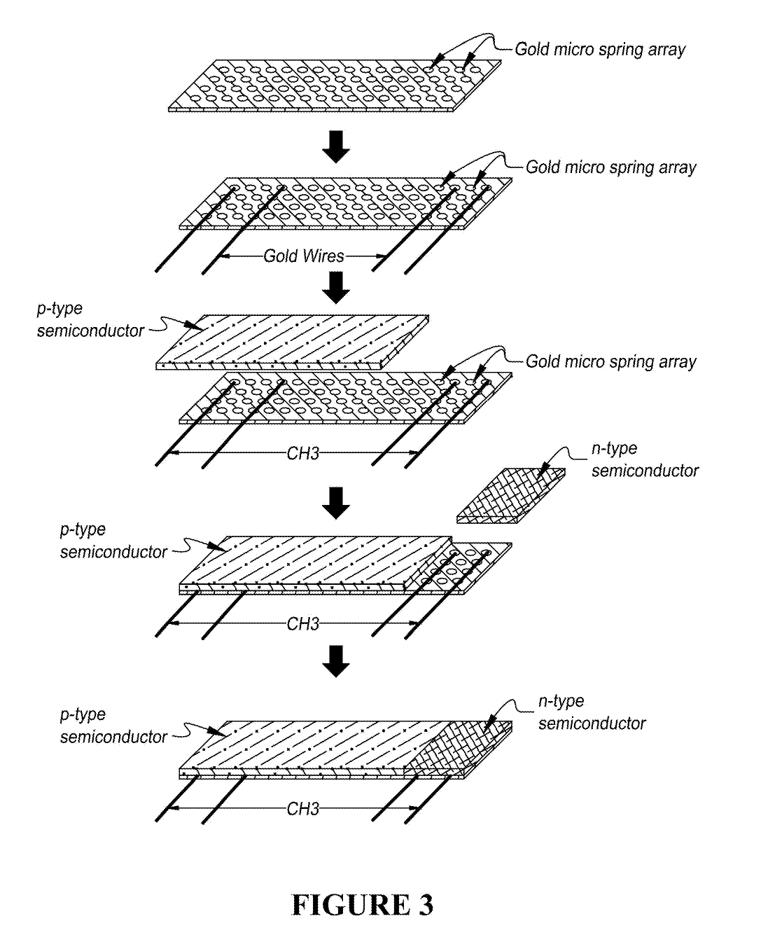

The procedures of sensor fabrication is shown in FIGS. 3A-D and 4A-D. The plastic substrate was washed by ethanol and distilled water. Gold wires were connected with the gold micro springs on the substrate. The commercial powders were ground thoroughly before use. 1 g of NiO powder was dispersed in 0.4 mL terpineol and blended into a thick slurry. 80 mg of the obtained NiO slurry was evenly painted onto the left side of the substrate. Then 1 g of In₂O₃ powder was mixed with 0.4 mL terpineol and 20 mg of the slurry was painted onto the right side of the substrate with a common interface. According to the area divided by vertical lines of four gold microsprings, the as-fabricated area ratio of the two semiconductors turned out to be 14:4 on the surface of the substrate (17.5 mm×4.5 mm). The substrate was designed in such a fashion that it has several leads at different distances, so that resistance across different lengths of the oxides could be measured. The sensor was calcined in air at 320° C. for 2 hours and kept in a tube furnace at 300° C. with flowing 20% O₂ in N₂ overnight before testing. The polymer substrate decomposed at 350° C., so alumina substrates of 10×10 mm with interdigitated gold lines of 0.25 mm spacing were used for high temperature measurements. After calcination at 320° C. in air for 2 h, the semiconductor layer was typically about 200 μm thick (discussed later).

Characterization

The phase and crystallinity of the metal oxides were analyzed by a Bruker D8 Advance X-ray diffractometer. The surface morphology of the sensor was investigated by a Quanta 200 scanning electron microscope. The chemical state of the metal oxides was examined by a Kratos X-ray photoelectron spectrometer with a mono Al source. The current-voltage measurement was performed on a CHI760D electrochemical workstation. The gas-solid interactions were studied by a PerkinElmer Spectrum 400 FTIR spectrometer coupled with a diffuse reflectance accessory. The Raman mapping of the interface was performed on a Renishaw-Smiths Raman Microprobe.

Gas Sensing Measurements

All gas sensing experiments were performed within a quartz tube placed inside a tube furnace (Lindberg/Blue) at 300° C., with a PC-controlled gas delivery system with calibrated mass flow controllers (Sierra Instruments INC.). The test gas mixtures containing different concentrations of NH₃ at constant oxygen content of 20 vol % were prepared by diluting NH₃ with O₂ and N₂. The total flow rate was maintained at 200 cm³/min. The resistance of the sensor was recorded by an Agilent 34972A LXI data acquisition/switch unit or a HP34970A at a scan rate of 0.1 Hz.

Human Breath Sensing Measurements

A system that simulates human breath with trace ammonia gas was developed. The system comprises a Mylar bag containing exhaled human breath samples and an ammonia gas cylinder. The trace ammonia gas at physiologically relevant concentrations was determined by controlling the flow rates of breath samples from the Mylar bags and ammonia supply, respectively. The total flow rate was maintained at 200 cm³/min. Three setups were designed. A first setup used a 37° C. water vapor bath to keep a constant humidity in the mixture of NH₃ and breath sample. The second setup used a dry ice/acetonitrile bath maintained at −20 to −25° C. to completely remove humidity in the mixture of breath+NH₃ and also an ice bath to reduce humidity. In both these setups, the breath sample was used as the background and NH₃ was spiked into the sample at increasing concentrations. In the third setup, air was used as the background, and the breath sample was measured, and then increasing amounts of NH₃ was added in, all gases passing through a moisture trap at −20 to −25° C.

Results

Characterization

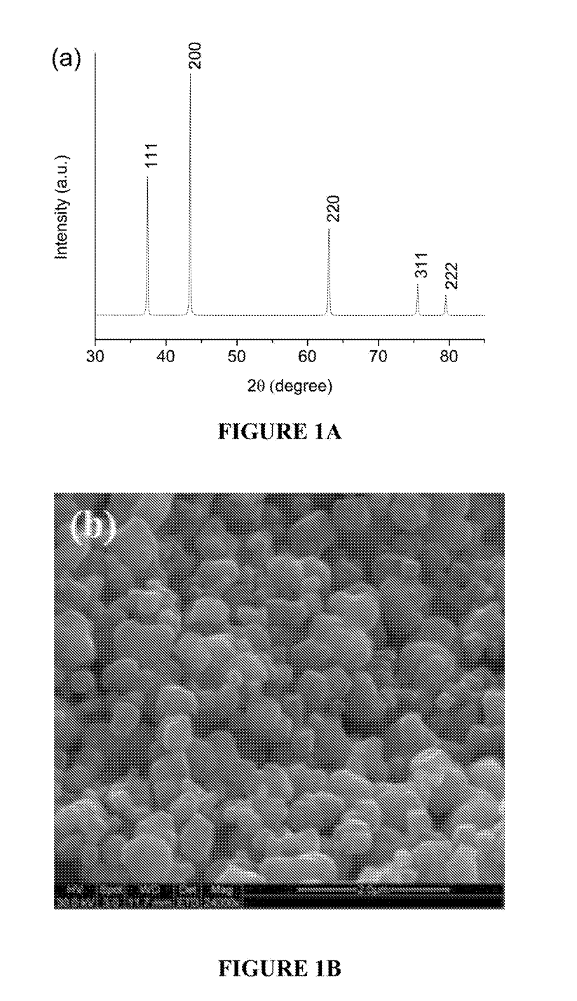

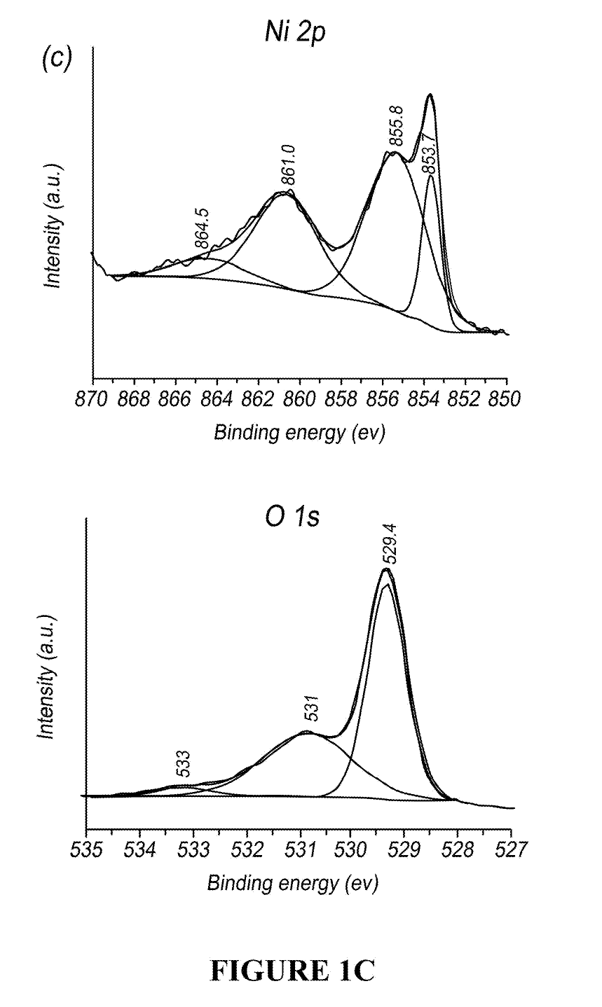

The two semiconducting oxides of interest in this study—NiO and In₂O₃—were obtained from commercial sources. Detailed characterization is presented in FIGS. 1A-C and 2A-C for NiO and In₂O₃ annealed at 320° C., respectively.

NiO:

The X-ray diffraction (XRD) pattern (FIG. 1A) is typical of the cubic structure of NiO (JCPDS No. 04-0835). Scanning electron microscopy (FIG. 1B) suggest particle diameters around 200-300 nm. X-ray photoelectron spectroscopy (XPS) of the O 1s region (FIG. 1C) suggest the presence of lattice oxygen (O²⁻, binding energy 529.4 eV), hydroxyl groups (binding energy 531 eV), and strongly chemisorbed oxygen (533 eV). In the nickel 2p_{3/2} region, peak at 853.7 is assigned to NiO₆ bulk cluster and peak at 855.8 eV to oxygen screened surface NiO₅ and nonlocal second neighbor screening of NiO₆ and NiO₅. The satellite region was fit to two peaks at 861.0 eV and 864.5 eV.

In₂O₃:

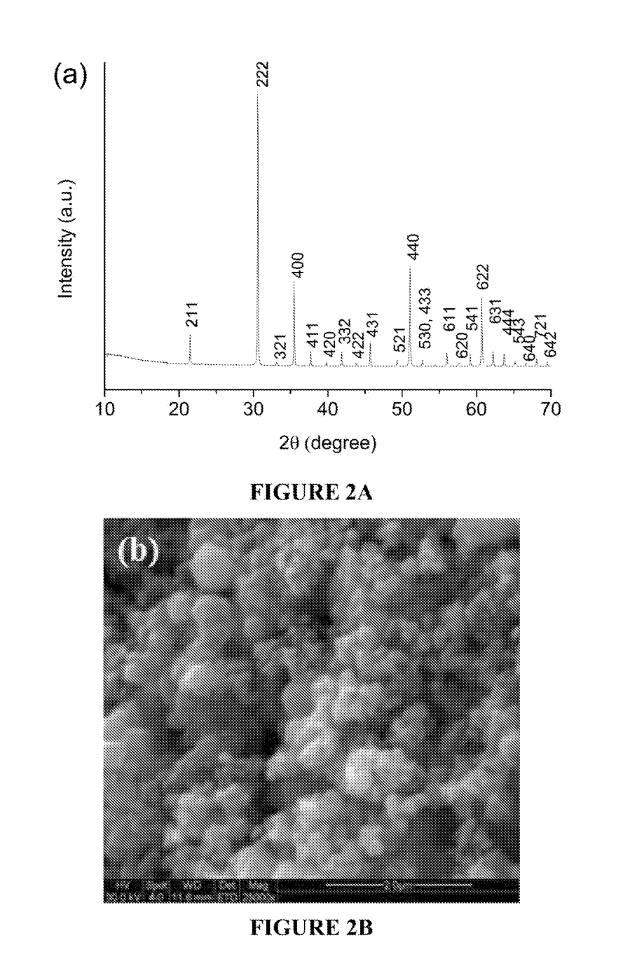

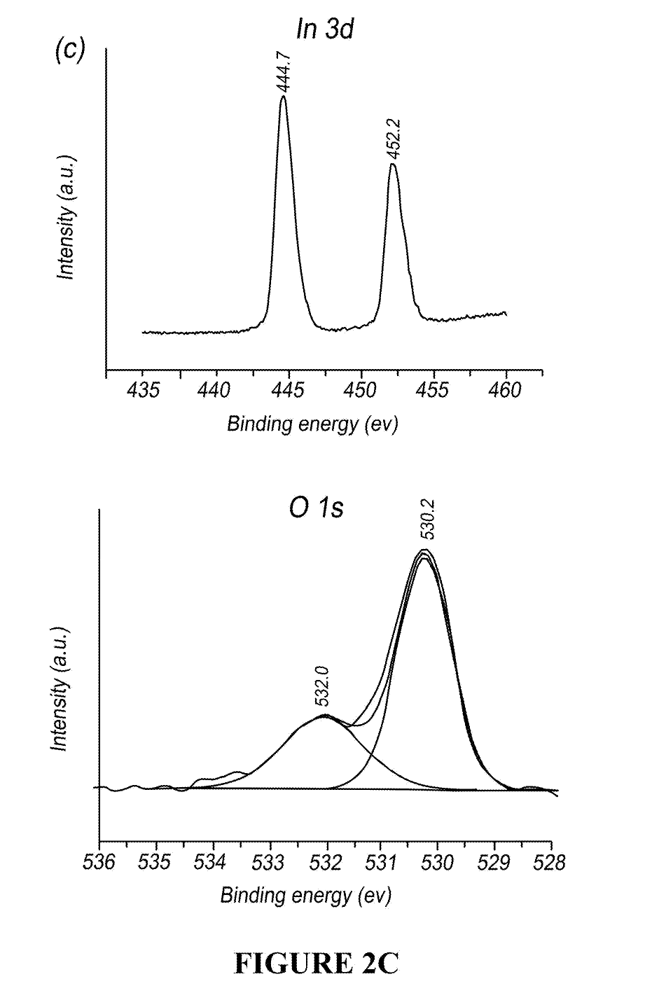

XRD pattern of In₂O₃ shown in FIG. 2A is indicative of cubic crystalline structure (JCPDS No. 06-0416). Size of the particles from the SEM micrograph (FIG. 2B) is <100 nm. XPS (FIG. 2C) indicates two peaks at 444.7 and 452.2 eV assigned to In 3d_{5/2} and 3d_{3/2} states, typical of In³⁺. The O 1s spectra is asymmetric with two peaks at 530.2 and 532.0 eV, with the former assigned to the oxygen lattice state, and the broad envelop at 532.0 eV to oxygen ions in oxygen deficient-regions (vacancies).

Sensor Characteristics

Design:

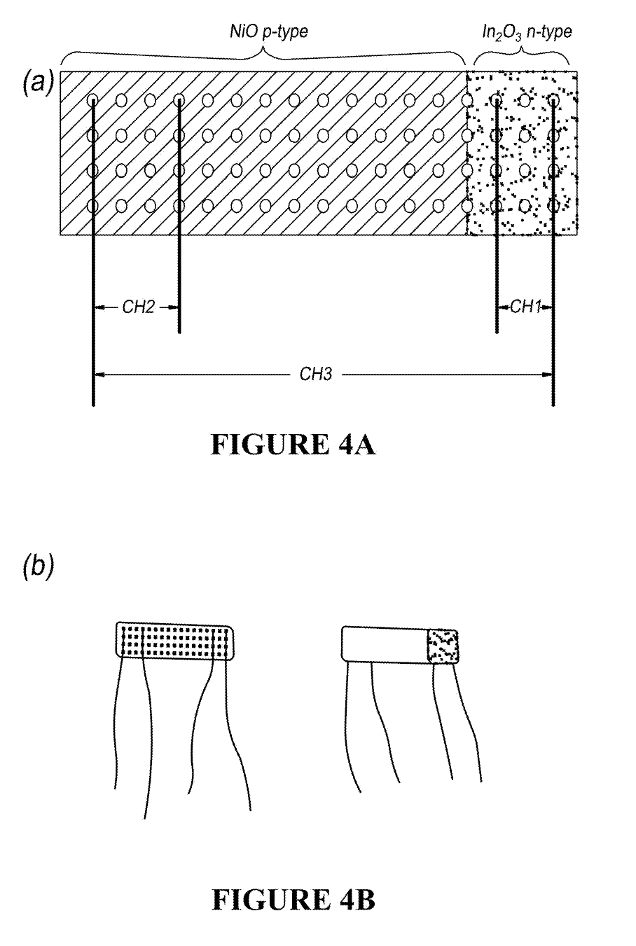

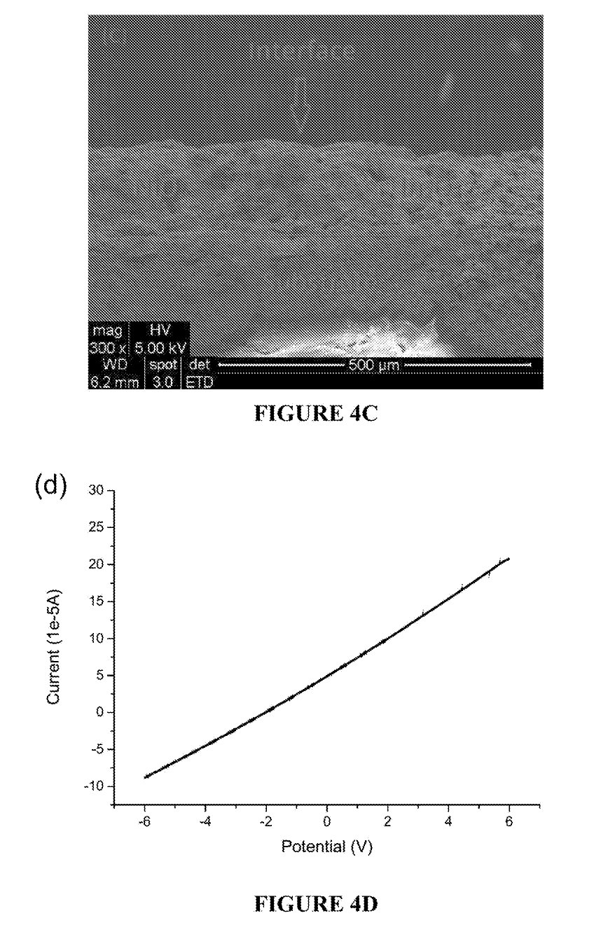

FIG. 3 shows a schematic of the steps involved in the sensor design, and FIGS. 4A-D show characteristics of the sensor. The two oxides are placed adjacent to each other on a plastic substrate and share a common interface. Substrate design makes it possible to measure resistances across varying lengths of the metal oxides (CH1 is defined as In₂O₃, CH2 as NiO and CH3 as a combination of both oxides, the choice of this combination is readily varied on the same sample). FIG. 4B shows a photograph of the sensor, with and without the oxide coating. The gold wires are used for the resistance measurements. FIG. 4C shows a side view of the sensor, indicating that the oxide films are ˜200 μm thick. These devices are heated at 320° C. in air for 2 hours, prior to making measurements at 300° C. FIG. 4D is the current-voltage (I-V) plot at 300° C. and displays a linear relationship, indicating there is no rectification, as expected for a diffuse mixing of the powders.

Microstructure:

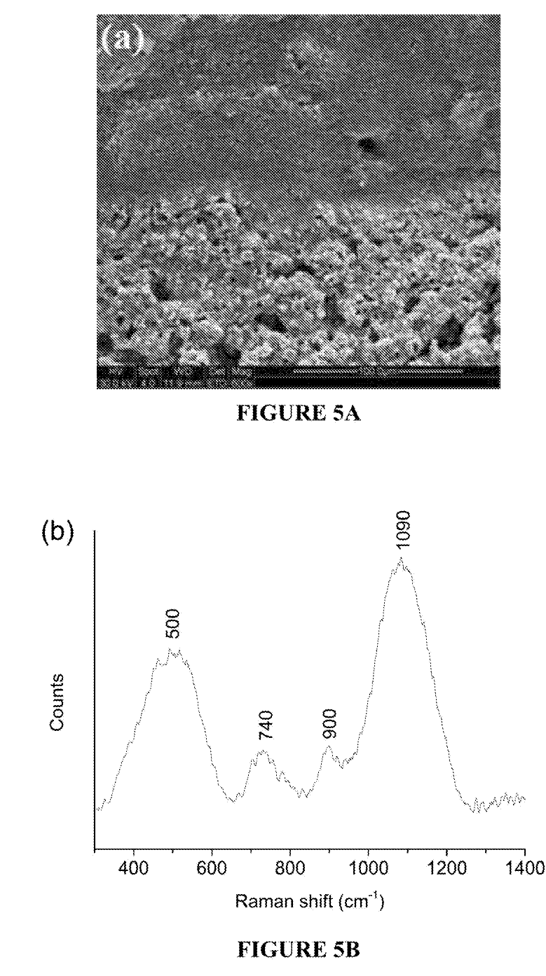

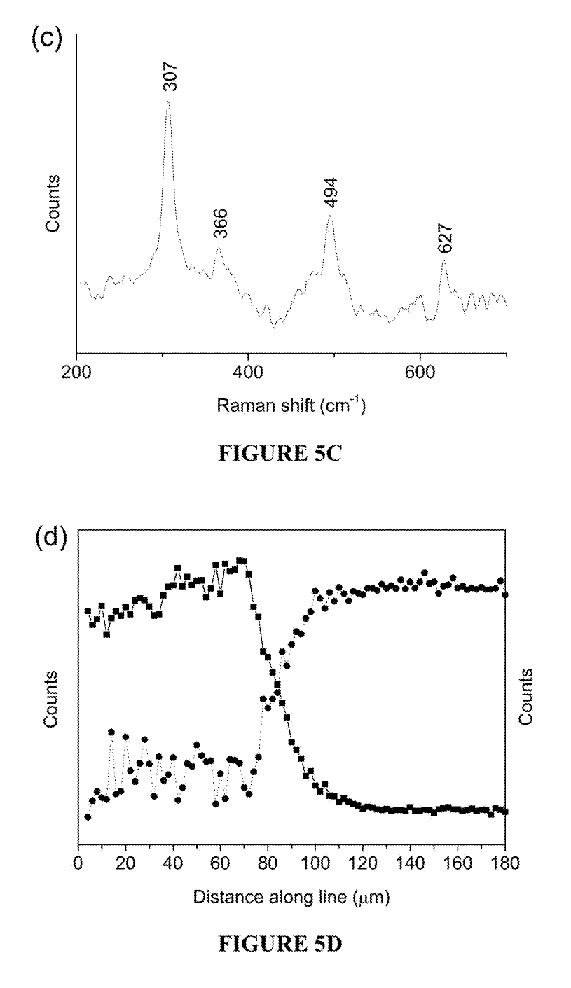

FIG. 5A shows the top-view SEM of the NiO/In₂O₃ interface. The NiO side of the sensor is characterized by Raman bands at 500, 740, 900 and 1090 cm⁻¹ (FIG. 5B), with the strongest bands at 500 and 1090 cm⁻¹, assigned to first and second order longitudinal optical modes, respectively. On the In₂O₃ side, bands observed at 307, 366, 494 and 627 cm⁻¹ (FIG. 5C) is consistent with previous literature. Raman spectra were recorded along a 180 μm length across the interface and the intensities of Raman bands of NiO (500 cm⁻¹) and In₂O₃ (307 cm⁻¹) are plotted in FIG. 5D. There is intermixing of the two oxides over a ˜30 μm distance at the interface.

Electrical Characteristics

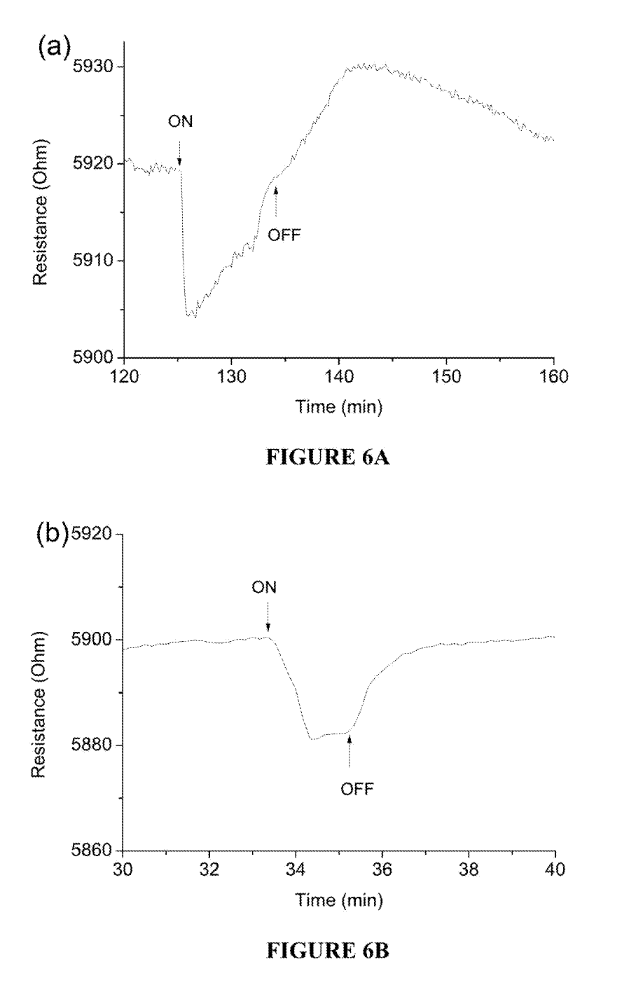

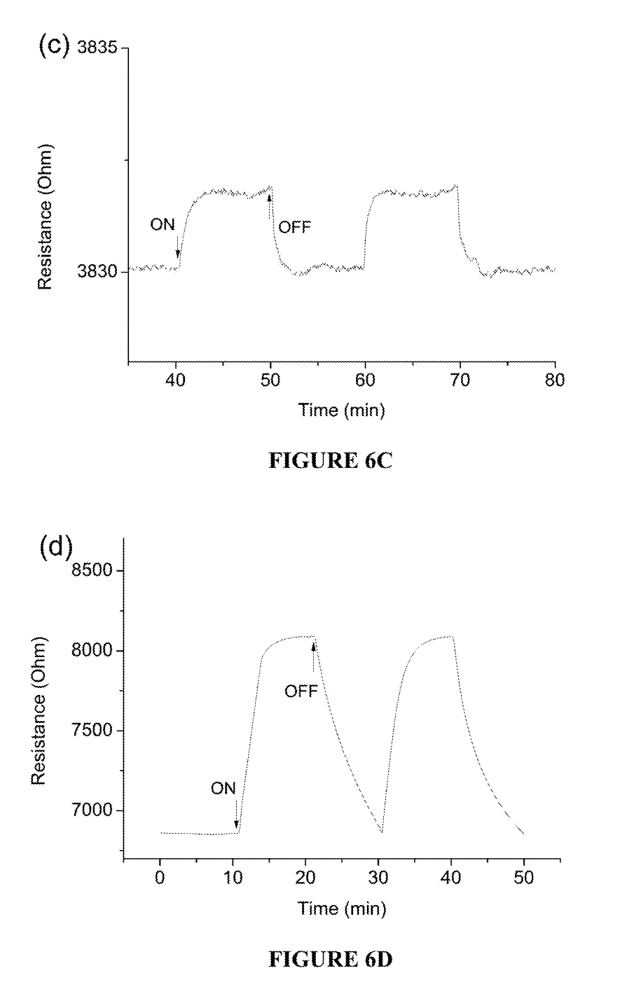

FIG. 6A is a plot of the resistance change of NiO after exposure to 1 ppm NH₃ at 300° C. With the gas pulse on, there is a decrease in resistance, followed by a slow increase. After the gas pulse is turned off after 10 minutes, the resistance continues to increase for 10 min (crosses the baseline), followed by a slow decrease to the baseline over the next 25 min. FIG. 6B shows that if the NH₃ gas pulse is only on for 2 min at 300° C., only a resistance decrease is observed, with both the response and recovery to the baseline occurring relatively rapidly (minutes). A 2 min exposure was used for all sensing experiments described later unless otherwise indicated. At a temperature of 500° C., the 1 ppm NH₃ registers a resistance increase (FIG. 6C). FIG. 6D shows an increase in resistance for 10 ppm NH₃ at 300° C.

Infrared Spectroscopy

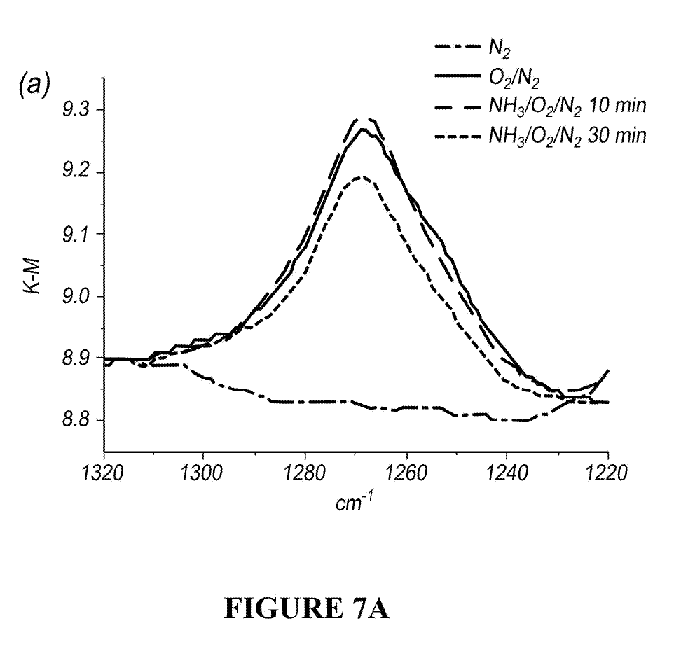

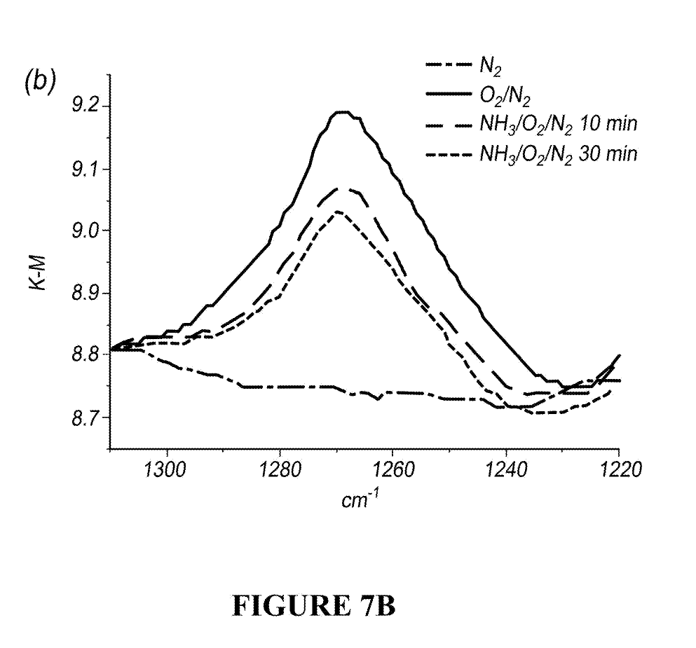

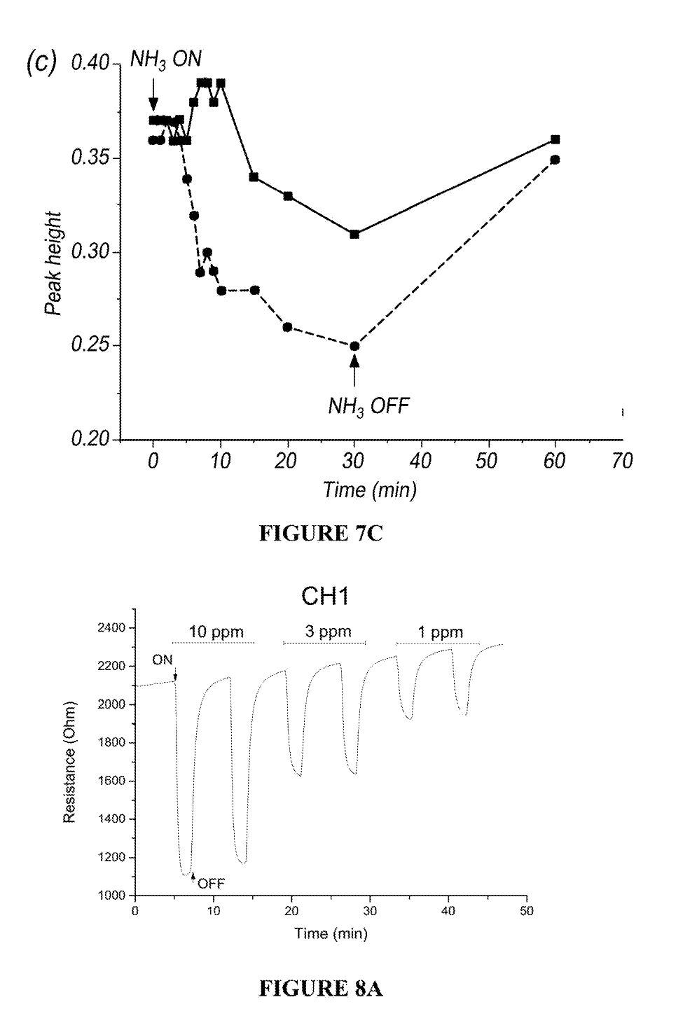

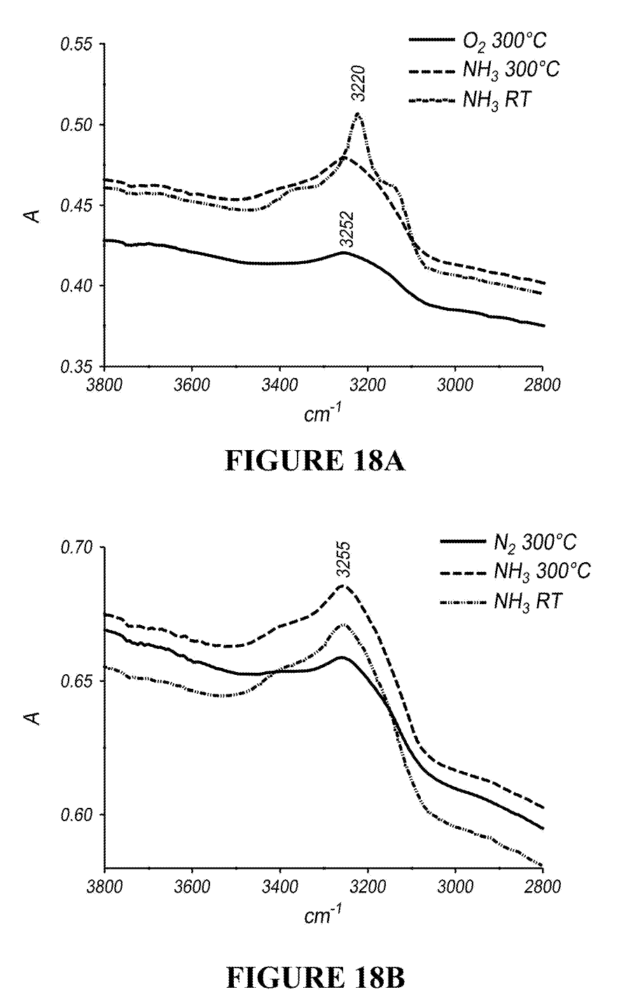

Infrared spectroscopy of the NiO surface was examined upon NH₃ exposure at 300° C. FIG. 7A focuses on the 1220-1320 cm⁻¹ spectral region, where changes with 1-10 ppm NH₃ were observed. With higher concentrations of NH₃ (100 ppm), a band at 3220 cm⁻¹ was observed in the presence of oxygen (FIGS. 18A-18B). With N₂ passing over the NiO sample, there is no band in the 1200-1300 cm⁻¹ region (FIG. 7A), but with 20% oxygen in the background gas, a band at 1267 cm⁻¹ appears. With 1 ppm NH₃, there is an initial increase in this band (10 min) followed by a gradual decrease (30 min), which is reversed upon the removal of NH₃ with 20% O₂. FIG. 7B shows spectral changes with 10 ppm NH₃, with the intensity of the 1267 cm⁻¹ band decreasing with time. FIG. 7C is a plot of the integrated intensity of the 1267 cm⁻¹ band versus time with 1 and 10 ppm NH₃. The increase in the intensity of the 1267 cm⁻¹ is obvious with 1 ppm NH₃, though with the 10 ppm, the intensity increase is not as clear, though the decrease in intensity of this band with time is more marked. Similar trends in resistance changes (FIG. 6A) and the intensity of the 1267 cm⁻¹ peak (FIG. 7A) are discussed in more detail below.

Sensing Characteristics

Carbon Monoxide:

All sensing experiments were done with 2 min pulses of the analyte gas. FIGS. 8A-8C show the behavior of the integrated NiO—In₂O₃ sensor (FIGS. 4A-4B) towards pulses of CO (10, 3, 1 ppm). Resistance across three channels are shown, comprising In₂O₃ (CH1, FIG. 8A), NiO (CH2, FIG. 8B) and the In₂O₃—NiO combination (CH3, FIG. 8C). With CO, the In₂O₃ shows a decrease in resistance (n-type behavior), and with NiO, an increase in resistance (p-type behavior). With the appropriate inclusion of both oxides, the change in resistance in the presence of CO is severely reduced.

Nitric Oxide:

FIGS. 9A-9C show the data with 5 and 10 ppb NO. Similar to the CO response, the NiO and the In₂O₃ show opposite responses (FIG. 9A-9B), but because NO is an electron acceptor, the direction of the resistance change is reversed, as compared to CO. Nevertheless, when the two metal oxides are combined (CH3), the response to NO is minimized (FIG. 9C).

Ammonia:

With NH₃ (1 ppm, 0.5 ppm, 0.1 ppm) on for a 2 min pulse, as shown in FIGS. 10A-10C, both In₂O₃ and NiO show a decrease in resistance, and when both oxides are included, the signal even at 100 ppb remains significant.

Mixture of Gases:

These experiments were then repeated with both NH₃ and CO in the gas stream with the 2 min pulses of gas. FIG. 11A-11C show the results. With In₂O₃, NH₃ (0.1, 0.5, 1 ppm) gives rise to a decrease in resistance (CH1, FIG. 11A). If CO (1, 3, 10 ppm) is included with the NH₃, the NH₃ signal is overwhelmed (CH2, FIG. 11B). Similar situation exists for NiO, except that a resistance increase is observed if CO is included in the gas pulse. However, the signal from the combination NiO—In₂O₃ channel (CH3, FIG. 11C) only exhibits a signal for NH₃, and the effect of CO, even at a concentration 100-fold higher than NH₃ is nullified.

Human Breath Samples

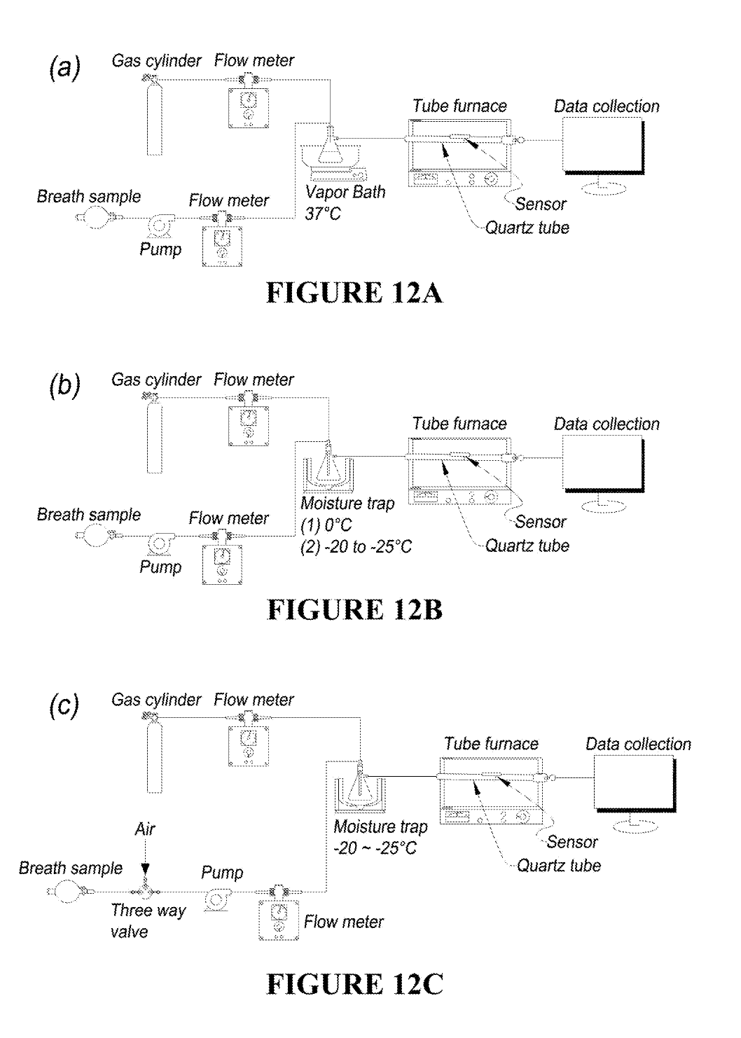

Three sets of experiments were carried out with human breath samples, and are schematically represented in FIGS. 12A-12C.

Using Breath as Background:

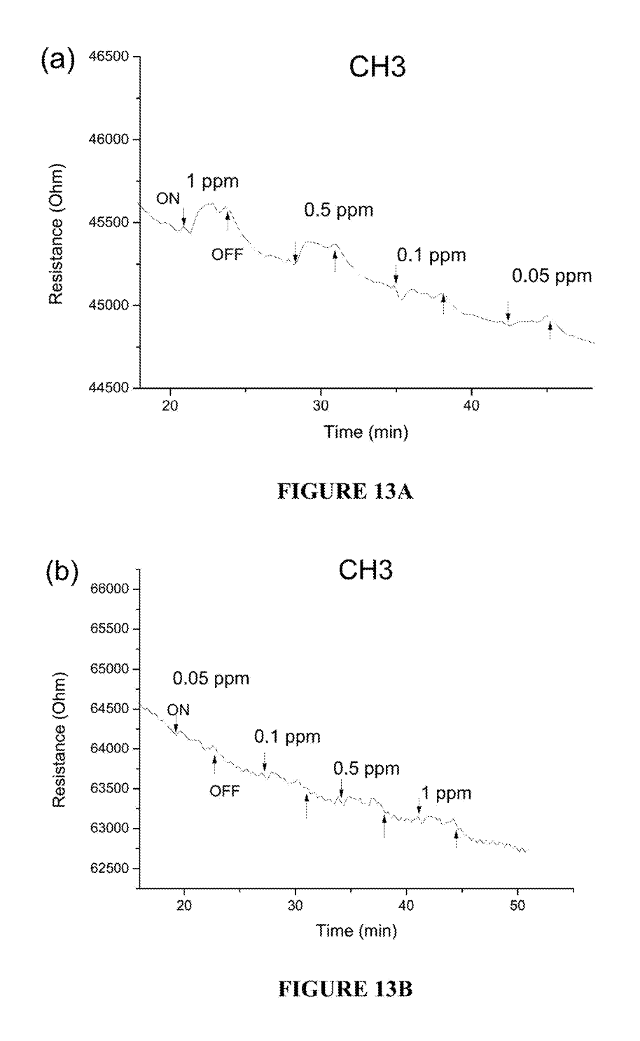

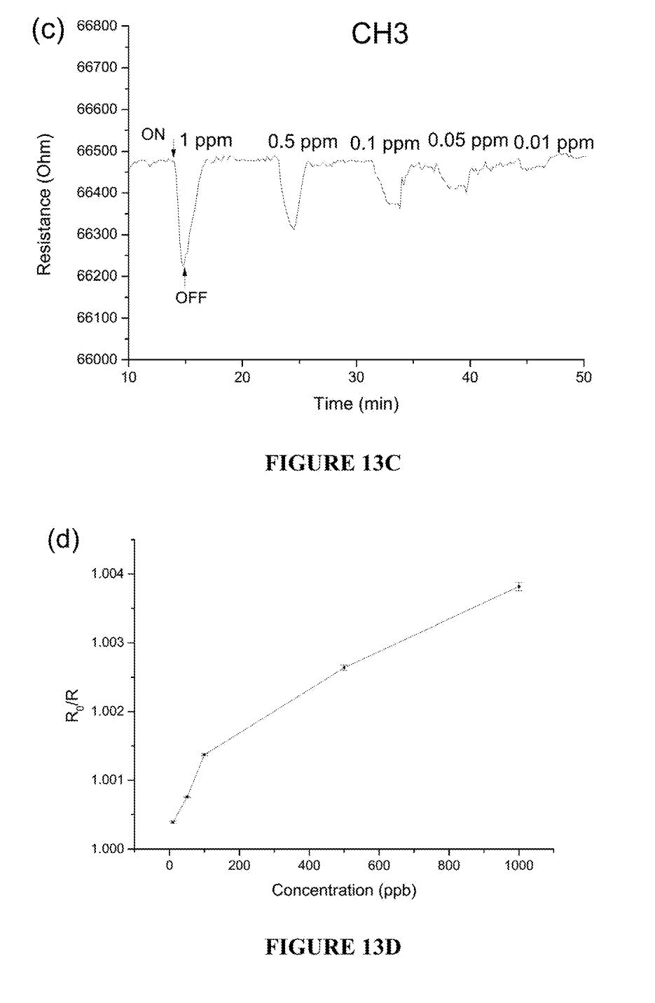

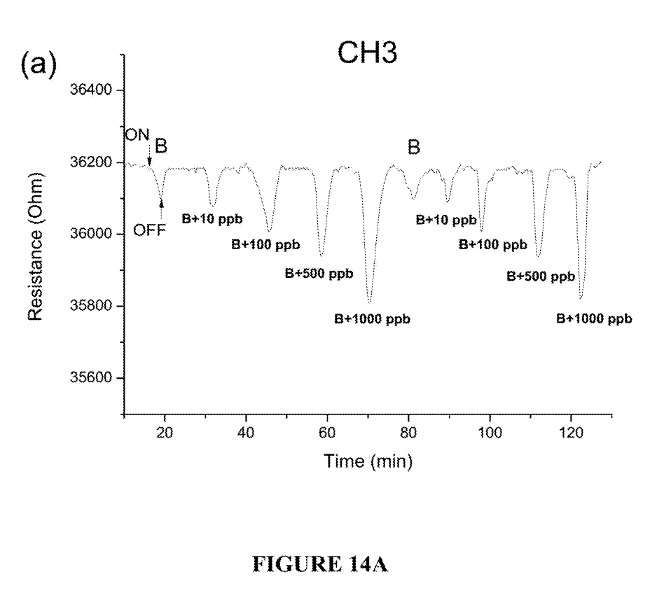

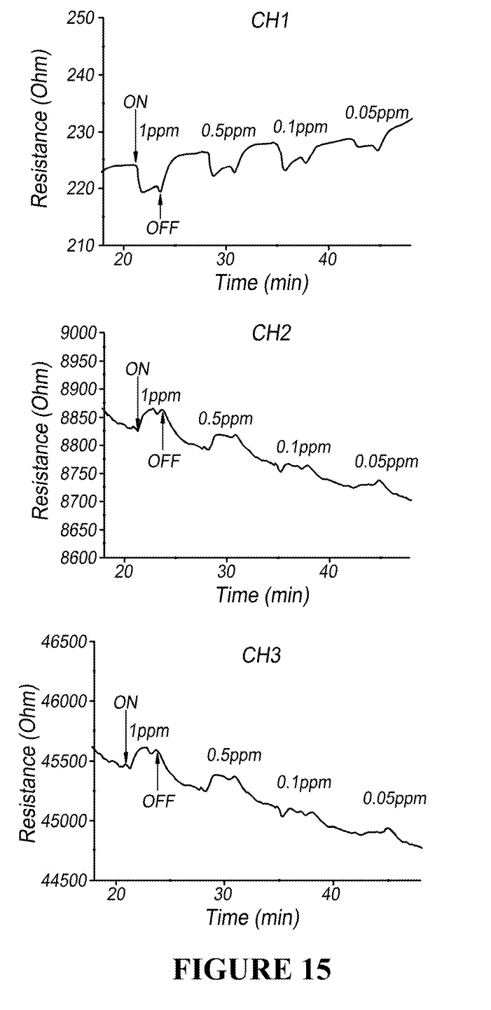

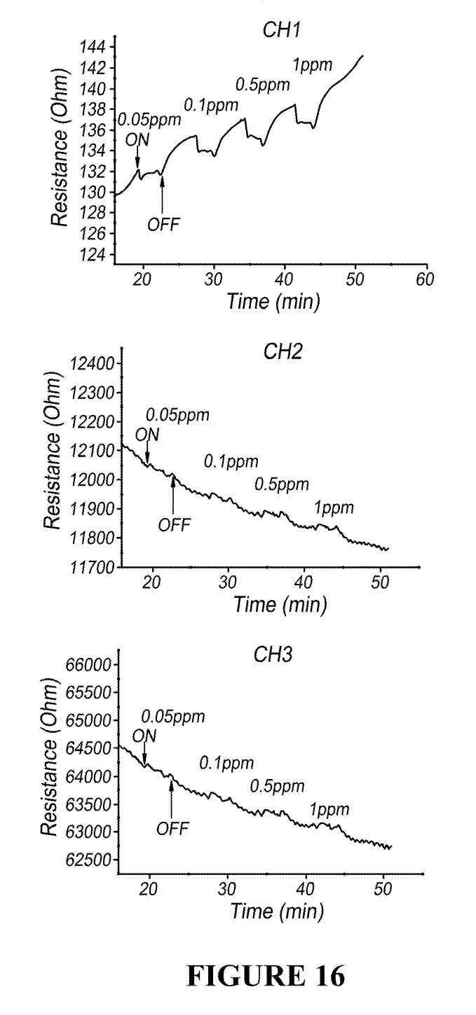

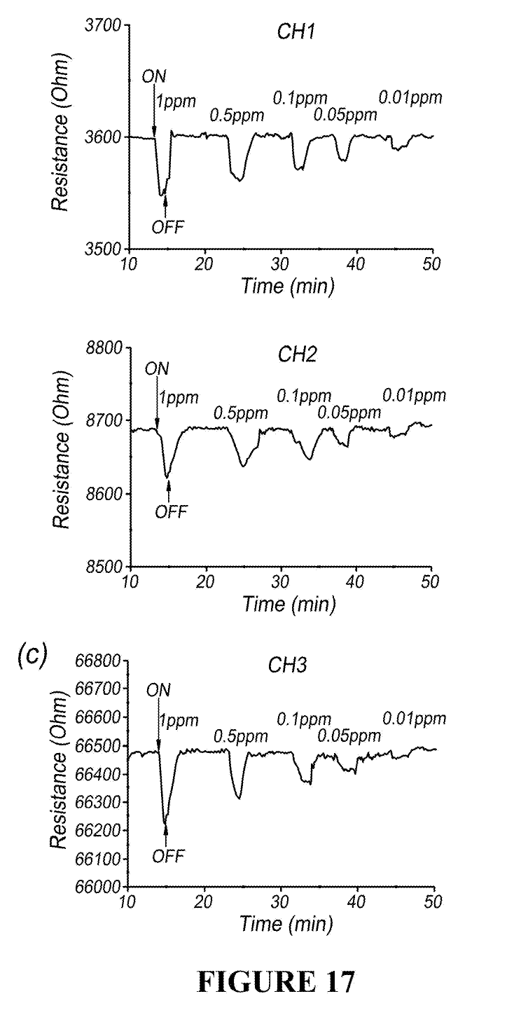

Breath samples were collected in Mylar bags. These samples were independently mixed via mass flow controllers with 10, 50, 100, 500, 1000 ppb of NH₃ and these samples were analyzed using the combined NiO—In₂O₃ sensor (CH3). In these experiments, the background signal was that of the breath alone, followed by introducing NH₃ in the gas mixture. The first experiment involved equilibrating the breath with water vapor at 37° C. with a measured relative humidity of 93% (FIG. 12A), followed by the sensing measurement. The second experiment involved passing the breath through an ice bath resulting in a humidity of 30% (using apparatus in FIG. 12B). The third experiment involved passing the breath through a moisture trap at −20 to 25° C., with a resulting humidity of 0% (FIG. 12B) The resulting sensing data using CH3 is shown in FIGS. 13A-13D (FIGS. 15-17 show the data for all channels). With both the humid samples (FIG. 13A-13B), the response to NH₃ was poor. The presence of water influenced the sensing signal of NH₃ on both NiO and In₂O₃, particularly the former (FIG. 15), with the NiO exhibiting a resistance increase with NH₃, the opposite of the observation with dry gas (FIGS. 10A-10C). With the breath sample mixed with NH₃ (bpt—33.7° C.) passing through the −20° C. trap, the expected signal to the spiked NH₃ was realized (FIG. 13C). The calibration curve with the breath sample is shown in FIG. 13D, and indicates saturation with increasing concentrations.

Using Air as Background:

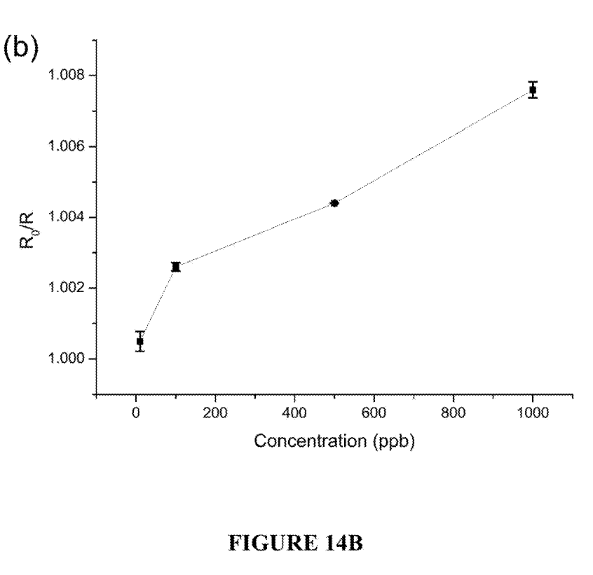

In another set of experiments, air was used as the background (FIG. 12C), and a breath sample was measured using CH3 (all samples passing through the dry ice trap at −20 to −25° C.). FIG. 14A shows that the breath alone provides a signal, though the species causing this signal cannot be ascertained. However, there was an increase in signal if the breath is mixed with NH₃, as shown in FIG. 14A. Such a standard addition experiment clearly indicates that the sensor is detecting NH₃. The background breath signal was normalized to Ro/R of 1, and the increased signal (measured as Ro/R) due to the spiked NH₃ is shown in FIG. 14B.

### DISCUSSION

In order to demonstrate the practical application of the sensor described in this paper, human breath sample was utilized as a proof-of-principle sample. The detection of NH₃ in human breath at ˜ppb levels could be helpful for diagnosis of various diseases. Typical levels for CO and NO in human breath are at ppm and ppb levels, respectively. The outcome of this study is a sensor that can detect NH₃ at low concentrations (<1000 ppb) with selectivity against CO at ppm and NO at ppb levels.

The sensor design employs a mixture of p- and n-type semiconducting oxide, but physically separated with a common interface (FIG. 3 and FIGS. 4A-4D). The separated p and n-oxides allows for altering the contribution of each oxide to the resistance more readily than physical mixture of powders.

The two oxides examined here are n-type In₂O₃ and p-type NiO. The conduction model for both n-type and p-type metal oxide gas sensors has been reviewed. In both n- and p-type oxides, oxygen ionosorption plays a key role in the sensing paradigm. In the case of n-type, such chemisorption leads to a decrease of majority carrier electrons at the surface of grains, whereas in p-type oxides, the oxygen ionosorption leads to a surface accumulation of holes. In n-type oxides, conduction is through the bulk of the oxide, whereas in p-type, conduction is along the surface. Under certain conditions, resistance changes from n- to p-type and vice versa has been observed. This effect is observed on Fe₂O₃, MoO₃, In₂O₃, SnO₂, TeO₂ and TiO₂, and several explanations have been proposed, including formation of a surface inversion layer driven via surface adsorption, different types of surface reactions, influence of polymorphs and morphology, as well as the effect of ionic dopants/impurities.

Resistance changes in NiO and In₂O₃ upon exposure to CO and NO were observed (FIGS. 8A-8B and FIGS. 9A-9B). NiO is behaving as a p-type semiconductor, with hole conduction as the main contribution. CO reacts with chemisorbed oxygen on the oxide surface releasing electrons, which raise the resistance of p-type NiO and lowers the resistance of n-type In₂O₃. With the appropriate contributions from both oxides, the resistance change to CO can be nullified (FIG. 8C). Similar observation are made with NO (FIG. 9C).

Under conditions in which NH₃ can react with chemisorbed oxygen, it usually behaves as a reducing gas, with proposed reactions such as:

2NH₃+3O⁻→N₂+3H₂O+3e (1)

2NH₃+5O⁻→2NO+3H₂O+5e (2)

These reactions are more favorable at higher temperature. The resistance changes upon interaction of NH₃ with metal oxides can be anomalous. For n-type oxides, such as In₂O₃ and WO₃, at lower temperatures (<300° C.), there is a resistance decrease. However at higher temperatures, initial resistance decrease is followed by a resistance increase. For n-type semiconductors, NO, the product of NH₃ oxidation upon chemisorption will lead to an increase in resistance. This competition between NH₃ oxidation and NO chemisorption is used to explain the anomalous sensing behavior. For avoiding the anomalous sensing behavior due to NOₓ, low temperature operation or the use of catalysts have been suggested. Other explanations for anomalous behavior, as in hexagonal-WO₃ have been ascribed to the formation of an inversion layer.

Our data on In₂O₃ at 300° C. indicates that NH₃ is behaving as a reducing gas (FIG. 10A), with a decrease in resistance. The resistance changes with NH₃ on p-type NiO is more complicated. The resistance increase observed 500° C. with 1 ppm NH₃ (FIG. 6C) and 10 ppm NH₃ at 300° C. (FIG. 6D) can be explained by reactions (1) and (2), where the electrons created upon NH₃ oxidation combine with the majority carrier holes, and lead to an increase in resistance, consistent with previous studies on NiO with 20-50 ppm NH₃. The behavior with 1 ppm NH₃ at 300° C. is not as expected and needs a different interpretation. As shown in FIG. 6A, there is an initial decrease in resistance over the first few minutes, followed by a gradual increase. Differences in direction of resistance changes as a function of analyte concentrations has been noted. On p-type TeO₂ at low temperatures (80° C.), a resistance decrease with ethanol (<300 ppm), an anomalous behavior, whereas with higher concentrations of ethanol, the resistance increased, as expected for a reducing gas on a p-type material. For p-type CuO nanowires, with NO₂ an oxidizing gas, at concentration <5 ppm, the resistance increased (anomalous behavior), whereas with 30-100 ppm NO₂, the resistance decreased, as expected for an oxidizing gas and p-type material.

The in situ IR spectra shown in FIGS. 7A-7C provide some clues. Formation of a band at 1267 cm⁻¹ on NiO is observed as gas is switched from N₂ to 20% O₂ at 300° C. (FIG. 7A-7C). This band disappears if the O₂ is replaced with N₂, so we assign this band to chemisorbed oxygen species. Upon introduction of 1 ppm NH₃, there is an increase in intensity of this band, followed by a decrease. The change in intensity of the 1267 cm⁻¹ band in FIG. 7C in the presence of 1 ppm NH₃ mirrors the change in conductivity upon 1 ppm NH₃ exposure to NiO, as shown in FIG. 6A (the timings do not exactly overlap since the IR was done on a powdered sample).

Several previous studies have noted a band in the 1200-1300 cm⁻¹ region upon oxygen chemisorption on metal oxides. On Fe₂O₃, bands between 1250-1350 cm⁻¹ have been assigned to perturbed O₂⁻ species, and in particular, the band at 1270 cm⁻¹ is prominent and stable up to 300° C. There are few infrared studies of oxygen adsorption on NiO, bands at 1070 and 1140 cm⁻¹ were observed at 77K and assigned to O₂⁻. On Fe₂O₃, bands in the 900-1100 cm⁻¹ were assigned to O₂²⁻ species. Formation of O0 on NiO has been proposed, though no distinct infrared bands were identified. Peroxo species (O₂²⁻) have been proposed upon oxygen adsorption on NiO. On CuCl and CuBr, a band around 1270 cm⁻¹ has been assigned to O₂ coordinated with Cu⁺, and intensity of this infrared band also decreased upon exposure to NH₃. Based on these studies, the band at 1267 cm⁻¹ (FIGS. 7A, 7B) on NiO can be assigned to O₂⁻.

The reactivity of NH₃ on metal oxide surfaces is enhanced in the presence of oxygen. On Mg (0001) surface, NH₃ was reactive with the surface only in the presence of oxygen. Chemisorbed oxygen on Ni (110) and Ni (100) is reactive with NH₃ with H abstraction and formation of NHₓ species. Surface spectroscopic studies have shown the high reactivity of NH₃ with adsorbed oxygen on Ni (111).

It has been proposed that the O₂⁻ is in equilibrium with O⁻:

O₂⁻ [symbol] O⁻+O (3)

NH₃ chemisorption at lower temperatures can lead to NH₂ and OH⁻ via reaction with the O⁻:

M^{x+} . . . NH₃+O⁻→M^{x+} . . . NH₂+OH⁻ (4)

Ammonia adsorption on alumina surface (acid/base sites) can lead to NH₂ and OH formation for about 10% of all the NH₃ molecules that are absorbed. Bands due to NH₂ were reported at 3386 and 3355 cm⁻¹. Dissociative chemisorption of NH₃ to NH₂ and OH driven by oxygen functionality is noted on epoxide groups in reduced graphene oxide, with vibrational bands assigned as 3208, 3270 cm⁻¹ (NH₂) and 3400 cm⁻¹ (OH). With the 1 ppm NH₃ on NiO, bands due to NH₂ were observed, but with 100 ppm NH₃ on NiO at 300° C., and subsequent cooling to room temperature, a band appears at 3220 cm⁻¹ in the presence of O₂, but not in the presence of only N₂ (these spectra are shown in FIGS. 18A-18B). The 3220 cm⁻¹ band can be assigned to N—H stretching.

Based on these observations, the anomalous behavior of 1 ppm NH₃ observed in FIG. 6A can be explained. It is hypothesized that reactions (3) and (4) take place on the NiO surface (we have IR evidence for the O₂⁻ species), and with (4) occurring, more O₂ chemisorption as O₂⁻, as O⁻ is used up in reaction (4) would be expected. IR indicates transient increase of the O₂⁻ band upon exposure to NH₃ (FIG. 7A). With increased O₂ chemisorption as O₂⁻, a decrease in resistance occurs. The increase in resistance observed later occurs due to subsequent reactions (1) and (2) due to oxidation of NH₃. At higher temperatures, or with higher concentrations of NH₃, the transient decrease in resistance is not observed (FIG. 6C, 6D) as reactions (1) and (2) are promoted.

The transient decrease in resistance upon exposure of low levels of NH₃ on NiO was exploited to amplify the sensor signal. This was done by exposing the NiO to only 2 min of NH₃, thus giving time for the chemisorption effects to occur (reactions 3 and 4, FIG. 6B), but not allowing time for the chemical reactions to occur (reactions 1 and 2). The resistance of NiO decreases and then recovers quite rapidly, as compared to the 10 min exposure, where the products of the chemical reaction form and need to be desorbed, before the sensor baseline is reached, and at 300° C., takes 40 minutes. With the 2 min NH₃ exposure, both NiO and In₂O₃ show a decrease in resistance, so the sensing data that combines both oxides (CH3) lead to an additive effect, amplifying the response from NH₃ (FIGS. 10A-10C) while with CO and NO, the opposite response lead to a cancellation of signal (FIGS. 8A-8C, 9A-9C). This strategy allows us to sense the presence of NH₃ in the concentration range of NH₃ in the concentration range <1000 ppb, in the presence of CO, as shown in FIGS. 11A-11C.

Since the need for detecting NH₃ in human breath is of the order of hundreds of ppb, breath samples were investigated as possible samples for use with this sensor. The high humidity in breath posed a significant interference (FIGS. 13A, 13B), and only upon removal of water from the breath by a cold trap (−20° C.), the signal due to NH₃ could be retrieved (FIG. 13C). Since NH₃ and H₂O can both act as Lewis bases, it is not surprising that humidity poses an interference to NH₃. Interference to humidity not only is present for NH₃, but to other gases such as CO, and for both n- and p-type materials. Chemisorption of water can follow the same as reaction (4), leading to formation of hydroxyl groups, with M^{x+}-OH bond formation. The observation that in the presence of water, there is an increase in resistance of NiO to NH₃ (FIG. 15) indicates that the water adsorption disrupts the oxygen chemisorption as O₂⁻, possibly by adsorbing at these sites. Thus, the p-n oxide arrangement can minimize interferences from other gases, such as CO, but because of the pronounced water interaction with the oxides, in general, humidity will be a strong interference to NH₃.

By removing the humidity, the sensor can detect NH₃ that is mixed into the breath. We have done the breath+NH₃ experiments in two ways. The breath is used as the background sample, and any increase in NH₃ in breath can be measured (FIGS. 13C-13D). Or, the breath can be measured using air as the background sample, the breath alone gives a signal and then any increase in NH₃ can be measured from the increased signal (FIG. 14A-14B). A possible biomedical application of this sensor would be measuring an increase of NH₃ in breath. For H. Pylori infection diagnosis, the current standard of measurement involves feeding the patient a sample of ¹³C- or ¹⁴C-labeled urea. The urease in the stomach (due to the bacteria) decomposes the urea to ¹³CO₂ or ¹⁴CO₂ and NH₃. The radioactive ¹⁴CO₂ in breath is then measured. With ¹³CO₂, a mass spectrometer is necessary to carry out the measurement. Because the sensor described herein can measure NH₃ at ppb levels, H. Pylori infection diagnosis could potentially be streamlined to involve feeding the patient regular (unlabeled) urea, and measuring the released NH₃. There would still be the need of the humidity trap to remove water. The trap can remove other organic volatiles in breath, but is not an issue in the application we are proposing for this sensor. Gases such as CO and NO (along with the NH₃) will still come through the trap, and the p-n strategy as outlined in this paper minimizes the influence of these interferents, while enhancing the signal for NH₃.

### CONCLUSION

This example demonstrates using p-type of NiO and n-type In₂O₃ placed side-by-side on a substrate with common interface as a sensor platform. The adjacent placement of the oxides allows for ease of variation of the amount of oxide to be included for making the resistance measurements in the presence of analyte gas. With this strategy, the change in resistance with 3-10 ppm CO was almost nil, since In₂O₃ and NiO give opposite responses to CO. Ammonia is also a reducing gas, but at low concentrations of NH₃ (<1 ppm) at 300° C., the response with In₂O₃ was a decrease in resistance, but with NiO, the resistance change was anomalous. For the first 8 min of a 10 min exposure to NH₃, there was a resistance decrease followed by a gradual resistance increase over the next 20 min, followed by a 10 min decrease to baseline resistance. With the help of in situ infrared spectroscopy, this behavior was correlated with NH₃ chemisorption and involvement of O₂⁻ species. Advantage was taken of the transient decrease with NH₃ on NiO to design a sensor that shows a resistance decrease for both NiO and In₂O₃ by controlling the gas pulses to a duration of 2 min. With this strategy, combining the two oxides enhanced the signal of NH₃, allowing ready detection at 100 ppb concentration. These sensors were used to detect NH₃ that was mixed with human breath. As long as the humidity is completely removed from the breath sample, 10-1000 ppb of added ammonia could be detected. Water interference arises from competing reactions with O₂⁻ and the transient decrease in resistance with NH₃ on NiO is no longer observed, thus removing the amplification. A potential application of such a sensor would be in H. Pylori diagnosis.

The devices, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A sensor device for sensing NH₃ in a gas sample, the sensor device comprising a sensing element comprising:
a first region comprising a p-type metal oxide semiconductor (MOS) material comprising NiO; and
a second region comprising an n-type MOS material comprising In₂O₃;
wherein the first region is adjacent to and contacts the second region.

2. The sensor device of claim 1, wherein the p-type MOS material consists of NiO.

3. The sensor device of claim 1 or 2, wherein the n-type MOS material consists of In₂O₃.

4. The sensor device of any of claims 1-3, wherein the sensor device further comprises:
a first electrode established within the first region;
a second electrode established within the second region; and
wiring interconnecting the first and second electrodes;
wherein a measured resistance along the wiring is indicative of the presence of NH₃ in a gas interfacing with the sensing element.

5. The sensor device of claim 4, further comprising a platform assembly maintaining the first and second electrodes as part of an electrode lead array selectively contacting the sensing element.

6. The sensor device of claim 5, wherein the platform assembly is configured to selectively alter a location of contact of the first electrode within the first region and selectively alter a location of contact of the second electrode within the second region.

7. The sensor device of claim 5 or 6, wherein the platform assembly is configured to selectively alter a distance between the first electrode and the second electrode.

8. The sensor device of any of claims 4-7, wherein a location of the first electrode relative to the first region and a location of the second electrode relative to the second region are selected such that the measured resistance is unaffected by the presence of CO, NO, or a combination thereof in a gas sample interfacing with the sensing element.

9. The sensor device of any of claims 4-7, wherein the sensing element defines a length from a first side to an opposing second side, the first side being defined by an edge of the first region opposite the second region, the second side being defined by an edge of the second region opposite the first region, and
wherein a location of the first electrode relative to the first region and a location of the second electrode relative to the second region are selected such that the wiring encompasses a combined amount of the p-type MOS material and the n-type MOS material in the length direction that is pre-determined to generate a measured resistance indicative of the presence of NH₃ in a gas sample interfacing with the sensing element.

10. The sensor device of claim 9, wherein the pre-determined combined amount is selected such that the measured resistance is unaffected by the presence of CO, NO, or a combination thereof in the gas sample interfacing with the sensing element.

11. The sensor device of any of claims 1-10, further comprising:
a third electrode established within the first region at a location separate from the first electrode;
a fourth electrode established with the second region at a location separate from the second electrode; and
wiring interconnecting the third and fourth electrodes;
wherein a measured resistance along the wiring interconnecting the third and fourth electrodes in comparison with the measured resistance along the wiring interconnecting the first and second electrodes is indicative of a concentration of NH₃ in a gas interfacing with the sensing element.

12. The sensor device of any of claims 1-11, wherein the p-type MOS material contacts the n-type MOS material at a diffuse p-n junction formed at an interface between the first and second regions.

13. A sensor system for sensing NH₃ in a gas sample, the system comprising a sensor device comprising:
a sensing element that comprises:
a first region comprising a p-type MOS material comprising NiO; and
a second region comprising an n-type MOS material comprising In₂O₃;
wherein the first region is adjacent to and contacts the second region,
a first electrode established within the first region;
a second electrode established within the second region; and
a database correlating measured resistance along wiring between the first electrode and the second electrode with presence of NH₃ in a gas sample interfacing with the sensing element.

14. The system of claim 13, wherein the p-type MOS material consists of NiO.

15. The system of claim 13 or 14, wherein the n-type MOS material consists of In₂O₃.

16. The system of any of claims 13-15, wherein the database further correlates an estimate of a concentration of NH₃ in the gas sample based upon the measured resistance.

17. The system of any of claims 13-16, wherein a location of the first electrode relative to the first region and a location of the second electrode relative to the second region is selected such that the measured resistance is unaffected by the presence of CO, NO, or a combination thereof in the gas sample.

18. The system of any of claims 13-17, wherein the database comprises a calibration curve.

19. The system of any of claims 13-18, further comprising a controller maintaining the database and electronically associated with the wiring.

20. The system of claim 19, wherein the controller comprises a memory on which is stored:
the database;
instructions for receiving a plurality of measured resistance values generated by the sensor device in the presence of the gas sample; and
instructions for estimating a concentration of NH₃ in the gas sample based upon the plurality of measured resistances.

21. The system of claim 20, wherein a first one of the plurality of measured resistances corresponds to a first distance between corresponding electrodes in the first and second regions, respectively, and a second one of the plurality of measured resistances corresponds to a second distance between corresponding electrodes in the first and second regions, respectively, the first distance being different from the second distance.

22. The system of any of claims 13-21, wherein the system is configured to estimate the concentration of NH₃ in human breath.

23. The system of any of claims 13-21, wherein the system is configured to estimate the concentration of NH₃ in a combustion gas.

24. A method of sensing NH₃ in a gas sample, the method comprising
providing a sensor system comprising:
a sensing element that comprises:
a first region comprising a p-type MOS material; and
a second region comprising an n-type MOS material;
wherein the first region is adjacent to and contacts the second region,
a first electrode established within the first region;
a second electrode established within the second region; and
a database correlating measured resistance along wiring between the first electrode and the second electrode with presence of NH₃ in a gas sample interfacing with the sensing element
contacting the sensor element of the sensor system with the gas sample,
measuring resistance along wiring between the first electrode and the second electrode, and
detecting NH₃ in the gas sample based upon the measured resistance.

25. The method of claim 24, wherein the p-type MOS material comprises NiO, CuO, Co₃O₄, Cr₂O₃, Mn₃O₄, or a combination thereof.

26. The method of claim 25, wherein the p-type MOS material comprises NiO.

27. The method of claim 26, wherein the p-type MOS material consists of NiO.

28. The method of claim 25, wherein the p-type MOS material does not include NiO.

29. The method of any of claims 24-28, wherein the n-type MOS material comprises In₂O₃, SnO₂, TiO₂, WO₃, ZnO, Fe₂O₃, or a combination thereof.

30. The method of claim 29, wherein the n-type MOS material comprises In₂O₃.

31. The method of claim 30, wherein the n-type MOS material consists of In₂O₃.

32. The method of claim 29, wherein the n-type MOS material does not include In₂O₃.

33. The method of any of claims 24-32, wherein detecting NH₃ in the gas sample comprises estimating a concentration of NH₃ in the gas sample based upon the measured resistance.

34. The method of any of claims 24-33, wherein a location of the first electrode relative to the first region and a location of the second electrode relative to the second region is selected such that the measured resistance is unaffected by the presence of CO, NO, or a combination thereof in the gas sample.

35. The method of any of claims 24-34, wherein the database comprises a calibration curve.

36. The method of any of claims 24-35, further wherein the sensor system further comprises a controller maintaining the database and electronically associated with the wiring.

37. The method of claim 36, wherein the controller comprises a memory on which is stored:
the database;
instructions for receiving a plurality of measured resistance values generated by the sensor device in the presence of the gas sample; and
instructions for estimating a concentration of NH₃ in the gas sample based upon the plurality of measured resistances.

38. The method of claim 37, wherein a first one of the plurality of measured resistances corresponds to a first distance between corresponding electrodes in the first and second regions, respectively, and a second one of the plurality of measured resistances corresponds to a second distance between corresponding electrodes in the first and second regions, respectively, the first distance being different from the second distance.

39. The method of any of claims 24-38, wherein contacting the sensor element with the gas sample comprises exposing the sensor element to the gas sample for a period of time effective to induce a decrease in the resistance of the p-type MOS material and a decrease in the resistance of the n-type MOS material.

40. The method of any of claims 24-39, wherein contacting the sensor element with the gas sample comprises exposing the sensor element to the gas sample for from 30 seconds to five minutes.

41. The method of any of claims 24-40, wherein contacting the sensor element with the gas sample comprises exposing the sensor element to the gas sample for from 1 to 3 minutes.

42. The method of any of claims 24-41, further comprising heating the sensor element to a temperature of from 250° C. to 450° C.

43. The method of any of claims 24-42, wherein the gas sample comprises a human breath sample.

44. The method of any of claims 24-43, wherein the gas sample comprises a combustion gas sample.

45. The method of any of claims 24-44, wherein the concentration of NH₃ in the gas sample is 5,000 ppb or less.

46. The method of any of claims 24-45, wherein the concentration of NH₃ in the gas sample is from 50 ppb to 2,000 ppb.

47. A sensor system for sensing NH₃ in a breath sample collected from a patient, the system comprising a sensor device comprising:
a sensing element that comprises:
a first region comprising a p-type MOS material; and
a second region comprising an n-type MOS material;
wherein the first region is adjacent to and contacts the second region,
a first electrode established within the first region;
a second electrode established within the second region;
a mouthpiece configured to collect the breath sample from the patient and deliver it into contact with the sensing element;
a database correlating measured resistance along wiring between the first electrode and the second electrode with presence of NH₃ in a gas sample interfacing with the sensing element;
a controller maintaining the database and electronically associated with the wiring, wherein the controller comprises a memory on which is stored:
the database;
instructions for receiving a plurality of measured resistance values generated by the sensor device in the presence of the breath sample;
instructions for estimating a concentration of NH₃ in the breath sample based upon the plurality of measured resistances;
instructions for assigning a score for the progression of an H. pylori infection in the patient based on the estimated concentration of NH₃ in the breath sample.
